(19) United States
(12) Patent Application Publication
(10) Pub. No.: US 2002/0182633 A1
(43) Pub. Date: Dec. 5, 2002

(54) Methods of patterning protein and cell adhesivity

(76) Inventors: Christopher S. Chen, Baltimore, MD (US); Joe Y. Tien, Brookline, MA (US); John Tan, Baltimore, MD (US); Sangeeta N. Bhatia, La Jolla, CA (US); William E. Jastromb, Baltimore, MD (US)

Correspondence Address: Dike, Bronstein, Roberts & Cushman, Intellectual Property Practice Group, EDWARDS & ANGELL, LLP, P.O. Box 9169, Boston, MA, 02209, US

(21) Appl. No.: 09/904,200

(22) Filed: Jul. 11, 2001

Related U.S. Application Data
(60) Provisional application No. 60/217,464, filed on Jul. 11, 2000.

(51) Int. Cl. B05D 3/00; C12Q 1/68; G01N 33/53

(52) U.S. Cl. 435/7.1; 427/2.11; 435/6

(57) ABSTRACT

The invention relates to a method of adhering a biomolecule to a substrate, comprising treating the substrate with a surfactant compound and a biomolecule. More particularly, the invention relates to a method of adhering a biomolecule to a substrate wherein the surfactant compound is not covalently linked to the substrate. The invention also relates to a device for adhering a biomolecule in a predetermined position comprising: a substrate having thereon a plurality of cytophilic regions that can adhere a biomolecule on the substrate by cytophobic regions to which the biomolecules do not adhere contiguous with the cytophilic regions, wherein the cytophobic regions comprise one or more surfactant compounds.

The present application claims the benefit of U.S. provisional application No. 60/217,464, filed Jul. 11, 2001, which is incorporated by reference herein in its entirety.

### STATEMENT OF GOVERNMENT SUPPORT

This invention was made with U.S. Government support including under Contact No. 335/98/S0465 funded by the Office of Naval Research. The Government has certain rights in the invention.

### FIELD OF THE INVENTION

The present invention relates to methods of spatially defining regions on a material surface to be adhesive or non-adhesive to proteins and cells, where the methods comprise treating the surface with a surfactant compound.

### BACKGROUND OF THE INVENTION

The ability to control cell-surface interactions is of paramount importance in controlling host-biomaterial interactions, in predicting cell behavior in cell engineering, in understanding tissue development, as well as in realizing the potential to tissue engineer solid organs. The role of tissue organization in many of these applications has been well studied and is ultimately modulated by receptor-mediated processes that influence cell behavior. The ability to control and study the role of tissue organization with micropatterning tools has recently provided insight in areas as diverse as: angiogenesis, hepatocyte differentiation, calicification of bone-derived cells, stratification of keratinocytes in the epidermis, and neuronal growth cone guidance [1-5].

Previous methods to create micropatterned cultures that control the cellular microenvironment have relied on either regional chemical modification of substrates to promote cell adhesion or physical localization of cells on a chemically uniform surface. Examples of chemical modification include photolithographic patterning of glass and subsequent silane/protein immobilization [6], microcontact printing to localize hydrophobic alkanethiols/protein [7], and photoimmobilization of polymers or adhesive peptides [8, 9]. Physical methods of localization include microfluidic networks to deliver adhesive proteins or live cells directly [10-12]. Similarly, laser-directed cell writing, is another method of physical localization utilizes a hollow optical fiber coupled with a laser source to direct the placement of individual cells on a target surface [13].

In photolithographic methods, adhesive proteins are typically localized by masking with light used for patterning of silanes that mediate adsorption of adhesive proteins such as vitronectin or immobilization of adhesive proteins such as collagen I [6, 14,15]. However, there are a number of drawbacks to using such approaches to modify biomaterials for cell adhesion. In particular, photolithography is commonly limited to rigid substrates (typically glass or silicon) that can withstand microfabrication processing (spinning, developing, lift-off) and typically only allows localization of a single chemical moiety (pro or non-adhesive). Thus, patterning of multiple distinct cell populations through a number of distinct ligands on a variety of biomaterials is difficult to achieve using photolithography alone. Previously, we have attempted to expand the utility of this tool by using it to localize 2 distinct cell types on glass- the first by selective adhesion to an adhesive protein (collagen I) and the addition of serum proteins with a second cell type which adsorb to the bare substrate and mediate cell adhesion of the second cell type to uncoated regions [16]. However, this approach cannot be expanded to 3 or more cell types without additional strategies. Thus, it would be desirable to have a method of localizing 3 or more cell types.

Alternatively, one can utilize a uniformly adhesive surface and localize cells spatially through use of a microfluidic network superimposed on the surface. One drawback of this system, however, is that resulting patterns must be spatially contiguous. In some cases, it is desirable to have isolated regions of cells (i.e. an island). In such cases, microfluidic cell delivery is not useful. Whitesides and coworkers [12] recently demonstrated that use of a laminated bilayer fluidic structure could indeed be used to create concentric isolated patterns of deposited proteins or cells. This methodology, however, requires a 4-step fabrication process and is only useful for cell types that will attach and grow within a microfluidic environment (e.g. oxygen and nutrient-limited).

Thus, it would be useful to have a method that enables the use of microfluidics to create isolated regions of cells and that is a simple, inexpensive process. It would be also useful to have a method of creating isolated islands and patterning of multiple cell types with a limited number of ligands in order to create more highly-structured multicellular culture environments.

The application of microfabrication to biology has resulted in several methods to produce microarrays of extracellular matrix to which cells can be attached. Most of these methods use photolithography, a light-based technique for patterning surfaces, to define regions on a substrate that cells could attach to, and regions that resist the attachment of cells. In general, these methods suffer from two drawbacks. First, because it is difficult to render a surface completely protein-resistant, often an initial pattern of proteins or cells breaks down over time: cells migrate in from regions that they adhere to, and simultaneously secrete proteins that facilitate the migration of surrounding cells. Second, the need to use specialized lithographic facilities every time in the production of each patterned substrate has limited the adoption of these techniques by biologists.

A series of soft lithographic approaches (i.e., using soft polymers as the basis for pattern generation) to pattern surfaces have been developed. One technique for patterning cells relies on non-lithography-based microscale printing of self assembled monolayers with an elastomeric stamp. (M. Mrksich, L. E. Dike, J. Tien, D. E, Ingber and G, M. Whitesides. Exp. Cell Res. 235, 305-313 (1997)). This technique is quick (it takes −1 min. to pattern a surface, −1 hr. to absorb a pattern of protein, and −1 hr. to seed a pattern of cells on it), cheap (the chemicals are either readily available or easily synthesized), and convenient (only an initial access to specialized facilities is needed). Alkanethiols spontaneously chemisorb from solution onto gold and silver to form ordered, oriented assemblies called self-assembled monolayers (SAMs); the functional properties of SAMs depend on the endgroup of the alkanethiol. (P. E. Laibinis and G. M. Whitesides. w-Terminated alkanethiolate monolayers on surfaces of copper, silver, and gold have similar wettabilities. J. Am. Chem Soc, 114,1990-1995 (1992).) These prior methods required the SAMs to be covalently linked to the substrate to ensure the durability of regions that resist the attachment of cells.

It would be desirable to have a method that enables the production of a patterned surface that does not require covalent linkage or other specialized materials or equipment.

### SUMMARY OF THE INVENTION

The present invention provides methods of spatially patterning surfaces to have areas that are adhesive, i.e., that will bind cells and other biomolecules and to have areas that are non-adhesive, e.g., are cytophobic areas.

More specifically, the invention relates to a method of patterning a surface with biomolecules comprising providing a non-adhesive agent to a portion of the surface, wherein the non-adhesive agent renders the portion of the surface inert to cell binding agents.

More particularly, in a first aspect, the invention provides methods for adhering a biomolecule to a substrate, which comprise treating the substrate with 1) a surfactant compound and 2) a biomolecule. Thereafter, the binding agent is applied to adhere the same to the binding agent and the substrate. Alternatively, a bioadhesive substrate can be utilized that would not require the use of a binding agent.

Significantly, the surfactant compound need not be covalently linked to the substrate for good performance results.

Preferred surfactant compounds for use in accordance with the invention comprise one or more hydrophobic regions and one or more hydrophilic regions. The surfactant compound suitably contains one or more hetero atoms, particularly one or more N, O or S atoms. Particularly suitable are surfactant groups that comprise alkoxy groups, such as alkoxy groups having one or more oxygen atoms and from 1 to about 20 carbon atoms per group. Alkylthio groups also are suitable, such as alkyl groups having one or more thio atoms and from 1 to about 20 carbon atoms per group. Shorter chain groups are generally preferred for hydrophilic regions of a surfactant compound such as alkoxy or alkylthio groups having 1, 2 or 3 carbons, more preferably 1 or 2 carbons, and loner chain groups are generally preferred for hydrophobic regions of a surfactant compounds, such alkoxy or alkylthio groups having 3 or more carbons, more typically 3, 4, 5, 6, 7, 8, 9 or 10 carbons.

Generally preferred surfactant compounds for use in accordance with the invention are polymeric materials, e.g. compounds having a molecular weight of at least about 500, 1000, 2000 or 3000, or even greater, such as at least about 5000, 6000, 80000, 10000, 20000, 30000, 400000 or 50000. Materials having a molecular weight in excess of about 200000 or 500000 may be less preferred for at least some applications.

Especially preferred polymeric surfactant compounds contain polyalkyl oxide groups (i.e. multiple alkoxy groups), such as polyC₁₋₂₀alkyl oxide units. Again, for hydrophobic regions of a surfactant, preferably longer chain units are employed, such as polyC₃₋₂₀alkyl oxide units, more typically polyC₃₋₁₂alkyl oxide units such as polypropylene oxide units. Shorter chain units are preferred for the hydrophilic units, such as polyethylene oxide units. The Pluronic or Tween polymeric are particularly suitable surfactant materials for use in accordance with the invention.

Surfactant compounds for use in accordance of the invention may comprise a variety of other groups, such as chargeable groups (e.g. carboxy; primary, secondary or tertiary amine), particularly on the hydrophilic surfactant regions. Preferably. The net charge of a hydrophilic regions is neutral, i.e. same number of each of anionic groups and cationic groups.

It also has been found that surfactant compounds can be imaged with selected radiation. This enables defining a desired pattern in a coating layer of surfactant compound and, in turn, selective, localized substrate adherence of a biomolecule.

In certain embodiments, prior to treating a substrate with a biomolecule, a binding agent is applied, such as a peptide. However, such a binding agent is not necessary if the biomolecule is capable of binding directly to the surface.

A wide variety of biomolecules may be adhered to a substrate in accordance with the present invention and include, e.g., peptides, polypeptides, nucleic acids, nucleic acid binding partners, proteins, receptors, antibodies, enzymes, carbohydrates, oligo saccharides, polysaccharides, cells, cell aggregagates, cell components, lipids, arrays of ligands (e.g. non-protein ligands), liposomes, microorganisms, e.g., bacteria, viruses, and the like.

A variety of substrates also may be employed as surfaces in accordance with the invention, including a variety of polymeric substrates, glass substrates, semi-conductirs, metals and the like. The substrate may have a variety of configurations such as slides, chambers and the like. The invention is particularly useful for microarray analysis, and the invention enables forming high concentrations of spatially segregated biomiolecules on a substrate surface, e.g. at densities of about 1 million biomolecules per cm² of the substrate surface, or higher densities such as 1.5 million biomolecules per cm² or 2million biomiolecules per cm² of the substrate surface. The invention also enables forming at least about 1 million spots per cm².

The invention also relates to the methods described above further comprising providing at least one additional and different biomolecule.

The invention also includes for adhering at least one cell or other biomolecule in a specific and predetermined position comprising: a surface, a plurality of cytophilic islands that adhere cells on said surface isolated by cytophobic regions to which cells do not adhere contiguous with said cytophilic islands, wherein said cytophobic regions are formed of a molecule having at least one hydrophobic region and at least one hydrophilic region. The surface of these devices surface comprises polymeric materials, PLGA, polyimide, polystyrene, glass, metal, and the like.

The cytophilic areas are created by the surface itself, or alternatively, by the immobilization of binding agents on the surface. Examples of binding agents include, but are not limited to proteins, e.g., fragments of compounds such as antigens, antibodies, cell adhesion molecules, extracellular matrix molecules such as laminin, fibronectin, collagen, integrin, serum albumin, polygalactose, sialic acid, and various lectin binding sugars, synthetic peptides, carbohydrates and the like.

Accordingly, a general purpose of the present invention is to provide an easily-synthesized or commercially available chemical species that readily adheres to a surface that is not chemically selective, and that prevents surface immobilization of a binding partner of a molecule desirably captured at the surface with a high degree of sensitivity and minimal to zero non-specific binding, in the presence of serum/fouling environments.

It is another purpose of the invention to provide an article with a surface that has a pattern of regions which have a high degree of sensitivity for a biological molecule and regions that do not bind the biological molecule.

Another purpose of the invention is to provide a method of capturing a biological molecule or a cell, by utilizing biological binding interactions that are extremely sensitive to molecular conformation and molecular orientation.

The present invention also provides a method of capturing a biological molecule or cell of interest. The method involves contacting a medium suspected of containing the biological molecule or cell with a solid phase that has a surface that binds the biological molecule or cell or has a plurality of binding agents that bind the biomolecule. The biological molecule then can be determined. According to one aspect the method involves providing a solid phase having a surface, and cytophilic regions on the surface separated from each other by cytophobic regions comprising a compound that is non-adhesive of the biological molecule or cell. According to this aspect the surface is brought into contact with a medium suspected of containing the biological molecule for a period of time sufficient to allow the biological molecule to bind to the surface.

The present invention also provides a kit including an article having a surface patterned with a non-adhesive agent and a binding agent, both as described above.

Other advantages, novel features and objects of the invention will become apparent from the following detailed description of the invention when considered in conjunction with the accompanying drawings.

### DESCRIPTION OF THE FIGURES

FIG. 1(a-b) show one method of the invention using direct printing to produce a surface patterned with protein and surfactant. FIG. 1(c) shows the surface after cells are seeded onto the surface in the presence of serum.

FIG. 2 (a and b) show one method of the invention that is used to control the pattern of hydrophilicity on surfaces by stamping patterns of hydrophobic and hydrophilic self-assembled monolayers of alkanethiols on gold.

FIG. 3 (a and b) shows one method of the invention that is used to pattern the surface of a substrate by masking the surface with a membrane.

FIG. 4 shows a schematic depiction of two modes of patterning. FIG. 4 (A) shows photolithographic patterning of glass substrates followed by immobilization of ‘adhesive’ (extracellular matrix proteins) or ‘non-adhesive’ (PEO) moieties. FIG. 4(B) shows lubrication of microfluidic PDMS mold to be utilized for delivery of cells, adhesive and non-adhesive moieties.

FIG. 5 shows fluidic localization of cells on photolithographically-patterned glass substrate. FIG. 5(A) shows a schematic depiction of method to localize hepatocytes through fluidic network on glass substrate patterned with collagen I islands. FIG. 5(B) shows a phase contrast micrograph of hepatocytes on 500 micron collagen I islands, localized within 2 mm networks. FIG. 5(C) shows fluorescent micrograph of cells in B. FIG. 5(D) shows a fluorescent micrograph of co-culture of repeating domains of micropatterned hepatocytes (green) and 3T3 fibroblasts (red). FIG. 5(E) shows individual composite island partially covered by both hepatocytes seeded through fluidic channel and fibroblasts seeded after removal of the PDMS network. Hepatocytes can be distinguished from fibroblasts by distinct nuclei and bright intercellular borders.

FIG. 6 shows fluidic localization of PEO adsorption to selectively deter cell adhesion on polystyrene. FIG. 6(A) shows a schematic of triblock (PEO/PPO/PEO) Pluronic™ FI08 molecule spontaneously adhering to a hydrophobic surface. FIG. 6 (B) shows that localization of 50 micron lane of PEO on (hydrophobic) polystyrene deters fluorescently-labeled 3T3 fibroblast cell adhesion in the presence of 10% serum. Triblock polymer spontaneously adsorbs to hydrophobic substrate via PPO core. FIG. 6(C) shows repulsion of fibroblasts at day 2, 10 and 14 in the presence of 10% serum in media.

FIG. 7 shows the characterization of pluronic F108-treated polystyrene substrates. FIGS. 7(A-F) show hepatocyte adhesion was assessed on (A) polystyrene control, (B) F108-treated polystyrene, (C) polystyrene coated with 100 ug/mL collagen I and F108-treated polystyrene coated with (D) 100, (E) 10 and (F) 1 ug/mL of collagen I. Adhesion was quantified by image analysis as seen in G.

FIG. 8 shows photolithographic and fluidic localization of PEO on hydrophilic substrates. In order to generalize adsorption of Pluronic F108 molecules to hydrophilic glass substrates, some wafers were first rendered relatively hydrophobic by grafting methyl-terminated silane to the surface. FIG. 8(A) shows a fluorescent micrograph of autofluorescent pattern of photoresist utilized to localize methylated silane modification in a donut shape. FIG. 8(B) shows a phase micrograph of previous surface, after grafting of methyl-terminated silane, removal of photoresist, and exposure to water. Note the array of water droplets retained by relatively hydrophobic annulus of methyl-terminated glass. FIG. 8(C) shows methyl-terminated micropatterns were utilized to pattern fibroblast adhesion; however, within 14 days, adsorption of serum proteins mediates migration of cells into previously bare regions. FIG. 8(D) shows adsorption of Pluronic F108 to hydrophobic methyl-terminated domains in C, in contrast, deterred cell adhesion for 14 days. FIG. 8(E) shows the results when the fluidic localization depicted in FIG. 2A was utilized to further localize Pluronic F108 deposition and fibroblast adhesion. FIG. 8(F) shows a low magnification view demonstrating patterning by specifying non-adhesive donut domains in contrast with adhesive domains utilized in 2C.

### DETAILED DESCRIPTION OF THE INVENTION

The following terms used herein are defined as follows. The term “biological binding” refers to the interaction between a corresponding pair of molecules that exhibit mutual affinity or binding capacity, typically specific or non-specific binding or interaction, including biochemical, physiological, and/or pharmaceutical interactions. Biological binding defines a type of interaction that occurs between pairs of molecules including proteins, nucleic acids, glycoproteins, carbohydrates, hormones and the like. Specific examples include antibody/antigen, antibody/hapten, enzyme/substrate, enzyme/inhibitor, enzyme/cofactor, binding protein/substrate, carrier protein/substrate, lectin/carbohydrate, receptor/hormone, receptor/effector, complementary strands of nucleic acid, protein/nucleic acid repressor/inducer, ligand/cell surface receptor, virus/ligand, etc.

The terms “binding agent”, “binding partner” “adhesive moiety” or “adhesive domain” refer to a molecule that can undergo biological binding with a particular biological molecule. For example, Protein A is a binding partner of the biological molecule IgG, and vice versa. Examples of these molecules are well known to those of ordinary skill in the art and include antigens, antibodies, cell adhesion molecules, extracellular matrix molecules such as laminin, fibronectin, synthetic peptides, collagen, carbohydrates and the like, as described herein. The term “adhesive” also refers to surfaces themselves which are capable of binding biological molecules or biomolecules.

The term “cytophobic” or “non-adhesive” refers to the surfactants described herein having a generally low affinity for binding, adhering, or adsorbing biological materials such as, for example, intact cells, fractionated cells, cellular organelles, proteins, lipids, polysaccharides, simple carbohydrates, complex carbohydrates, and/or nucleic acids. These surfactants are described in greater detail below.

The terms “biological molecule” or “biomolecule” refers to a molecule that can undergo biological binding with a particular biological binding partner. For the purposes of this application, the term “biological molecule” and “biomolecule” also refers to living materials, e.g., cells, microorganisms, viruses, etc. Examples include, e.g., peptides, polypeptides, nucleic acids, nucleic acid binding partners, proteins, receptors, antibodies, enzymes, carbohydrates, oligo saccharides, polysaccharides, cells, cell aggregagates, cell components, lipids, arrays of ligands (e.g. non-protein ligands), liposomes, microorganisms, e.g., bacteria, viruses, and the like.

The term “recognition region” refers to an area of a binding partner that recognizes a corresponding biological molecule and that facilitates biological binding with the molecule, and also refers to the corresponding region on the biological molecule. Recognition regions are typified by sequences of amino acids, molecular domains that promote van der Waals interactions, areas of corresponding molecules that interact physically as a molecular “lock and key”, and the like.

The term “non-specific binding” (NSB) refers to interaction between any species, present in a medium from which a target or biological molecule is desirably captured, and a binding partner or other species immobilized at a surface, other than desired biological binding between the biological molecule and the binding partner.

The term “self-assembled monolayer” or “SAM” refers to a relatively ordered assembly of molecules spontaneously chemisorbed on a surface, in which the molecules are oriented approximately parallel to each other and roughly perpendicular to the surface. Each of the molecules includes a functional group that adheres to the surface, and a portion that interacts with neighboring molecules in the monolayer to form the relatively ordered array. See Laibinis, P. E.; Hickman, J.; Wrighton, M. S.; Whitesides, G. M. Science 245, 845 (1989), Bain, C.; Evall, J.; Whitesides, G. M. J. Am. Chem. Soc. 111, 7155-7164 (1989), Bain, C.; Whitesides, G. M. J. Am. Chem. Soc. 111, 7164-7175 (1989), each of which is incorporated herein by reference.

The present invention provides a method for producing patterned surfaces for defining cells, proteins, or other biological materials in a specific and predetermined pattern. In particular, it provides a method of producing surfaces with patterned regions of binding, e.g., material capable of binding biological molecules, cells, proteins or other biological materials, interspersed with non-adhesive regions, e.g., material that prevents the adhesion of the biological molecule or cell to the surface. The present invention provides for the production of patterned surfaces in which the dimensions of the features or details of the patterns may be smaller than 1 μm.

The invention derives from a general new method of creating patterned surfaces applicable in a variety of fields. The method is simple and provides for relatively inexpensive production of many copies of the patterned surface.

The patterns of binding regions and/or non-adhesive regions of the present invention are formed by modification of known methods, e.g., stamping, microfluidics, photolithography, microcontact printing, nanopen lithography, subtraction active devices, eletrophoresis, etc., and unique combinations thereof as described herein. For example, a protein that will be used to bind cells can be applied to the surface using a stamp in a “printing” process in which the “ink” consists of a solution including a compound capable of binding the cells. The “ink” is applied to the surface using the stamp and deposits the protein on the plate in a pattern determined by the pattern on the stamp. The surface may be stamped repeatedly with the same or different stamps in various orientations and with the same or different proteins. The general process of stamping is described in U.S. Pat. No. 5,776,748, which is incorporated herein in its entirety.

The methods of the present invention relate to the novel use of a surfactant as a non-adhesive agent on the portions of the surface, e.g., a plate, which remain bare or uncovered by a binding agent, to prevent binding of protein or cells to the surface. Thus, patterns can be created on the surface of binding areas and non-binding areas. For example, a pattern of islands may be created in which the islands of the grid are cytophilic, i.e., bind cells, but the regions around the islands are cytophobic and no cells bind to these regions.

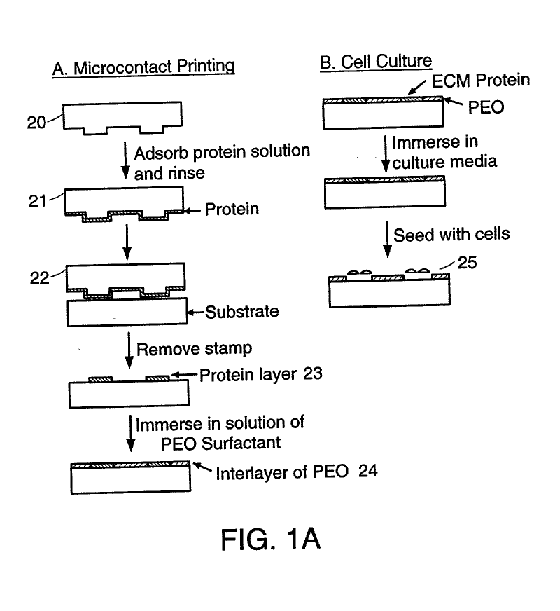

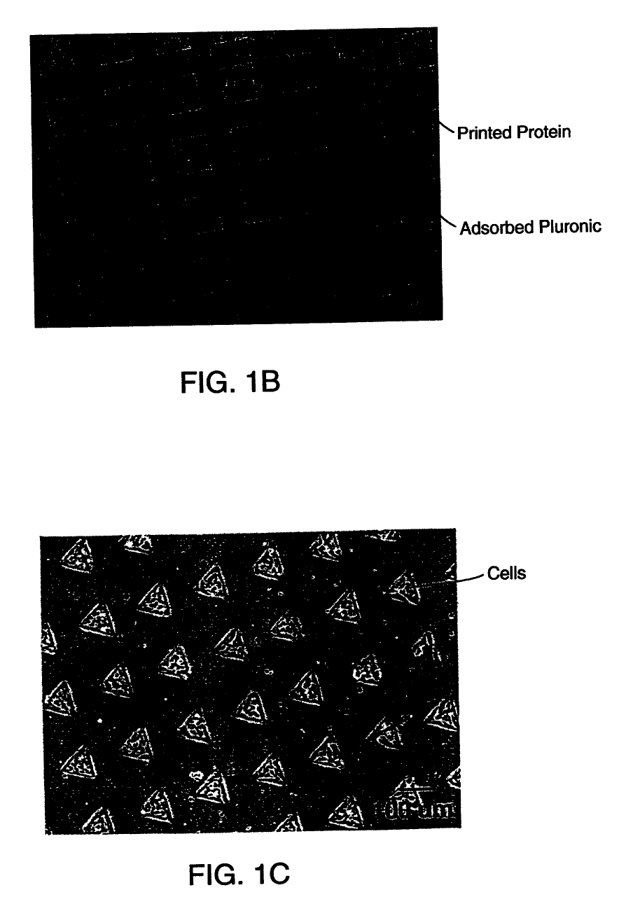

A simple illustration of the general process of stamping using the present invention is presented in FIG. 1. FIG. 1(a) shows the process of printing and cell culture. A stamp 20 is manufactured, e.g., by casting a polymeric material onto a mold with raised features defining a pattern. The stamp is “inked” with a protein 21. Upon rinsing, the stamp is microcontact printed onto a surface or substrate of choice under ambient conditions 22. When the stamp is removed, a protein layer 23 remains on the substrate. Upon rinsing, if necessary, the non-adhesive agent is then allowed to adsorb onto the surface to block areas not printed with protein 24, see also FIG. 1(B). Because the hydrophobic core of the nonadhesive agent is responsible for its stable adsorption onto the surface, it is unable to adsorb to the protein-adsorbed, hydrophilic areas. The surface patterned with the cell binding agent, i.e., protein, and cell non-adhesive agent is then immersed in culture media and seeded with cells 25 in the presence of serum. Cells they selectively attach to the areas where the adhesive protein is printed (see FIG. 1c). To produce surfaces with arrays of cells, the protein printed onto the surface is one that cells can adhere to, usually a member of the extracellular matrix family of proteins.

While the process of stamping is described in detail herein, it is intended that the present method of using a surfactant as described herein as a non-adhesive agent, can be used in many processes for patterning cells. Further examples are shown and described below.

As aforesaid, the non-adhesive agents of the present invention comprise compounds that have at least one hydrophilic region and at least one hydrophobic region. Examples of compounds that are useful as non-adhesive agents include surfactants. As discussed above, specific examples of useful surfactants include Pluronics F127, P105, P123, and Tween-20.

The methods of the present invention are advantageous over existing methods to pattern proteins and cells in several aspects. First, in the methods of the present invention, the binding agents, i.e., proteins, patterned using soft lithography are never exposed to harsh solvents that may denature and change the conformation as well as the function of the binding agent. Second, these present methods are compatible with a wide range of surfaces ranging from various polymeric, glass, and evaporated metal surfaces.

The following are examples of different methods of producing patterns using the methods of the present invention. In a direct printing method, to produce an array of protein patches, the protein of choice is allowed to adsorb onto a microfabricated elastomeric stamp (see FIG. 1a). Upon rinsing, the stamp can be microcontact printed onto a surface of choice under ambient conditions. Upon rinsing, a surfactant, e.g., Pluronics is then allowed to adsorb onto the surface for 1 hr to block areas not printed with protein (see FIG. 1b). Because the PPO hydrophobic core of Pluronics is responsible for its stable adsorption onto the surface, it is unable to adsorb to the protein-adsorbed, hydrophilic areas.

To produce surfaces with arrays of cells, the protein printed onto the surface is one that cells can adhere to, usually a member of the extracellular matrix family of proteins. Cells are then seeded onto the surface in the presence of serum and they selectively attach to the areas where the adhesive protein is printed (see FIG. 1c). This method allows the patterning of Pluronics and thus areas that will resist protein adsorption and cell attachment.

Proteins and cells have been successfully patterned onto PDMS, oxidized PDMS and polystyrene surfaces using this method.

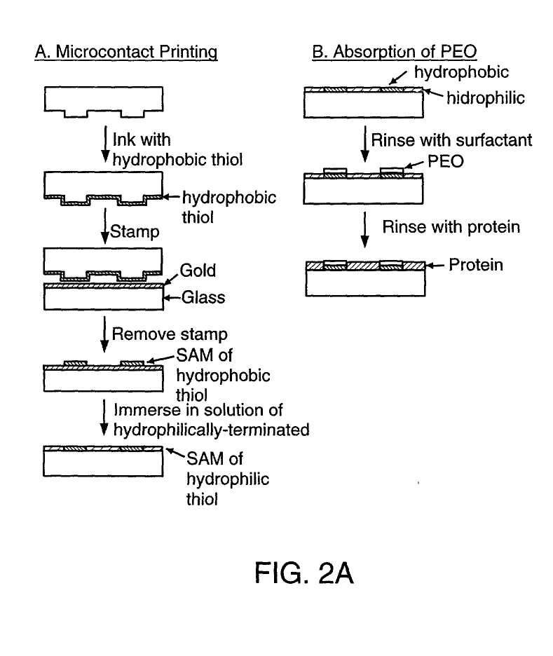

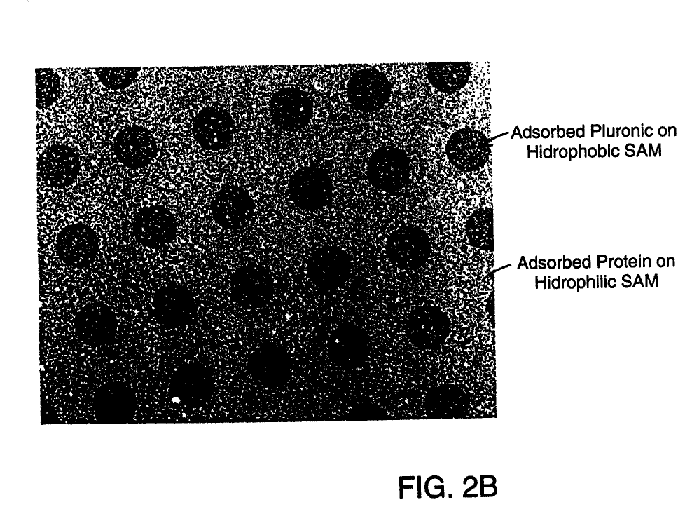

In another embodiment, the pattern of hydrophilicity on surfaces can be controlled by using combinations of materials having different hydrophilicity, e.g., by stamping patterns of hydrophobic and hydrophilic self-assembled monolayers of alkanethiols on gold. In this procedure, a hydrophobic-terminated alkanethiol is stamped onto the surface and then the surface is rinsed with a hydrophilic-terminated alkanethiol, on gold-coated substrates. By rinsing with the PEO surfactant, the PEOS will selectively adsorb to the stamped, hydrophobic regions. Coating with protein subsequently coats the hydrophilic regions (see FIG. 2).

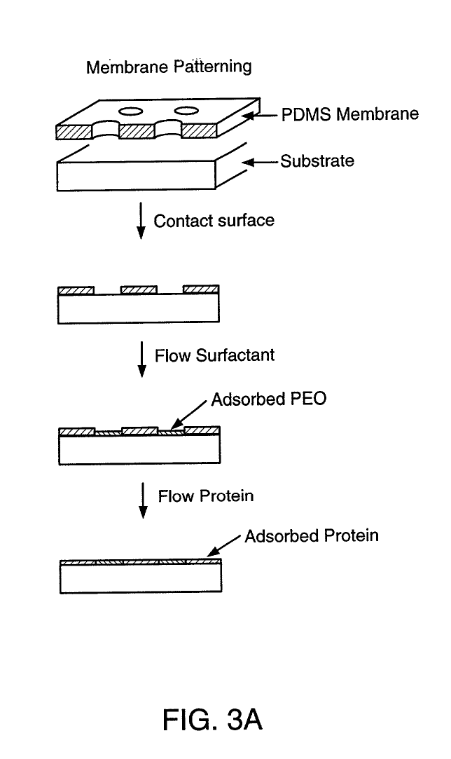

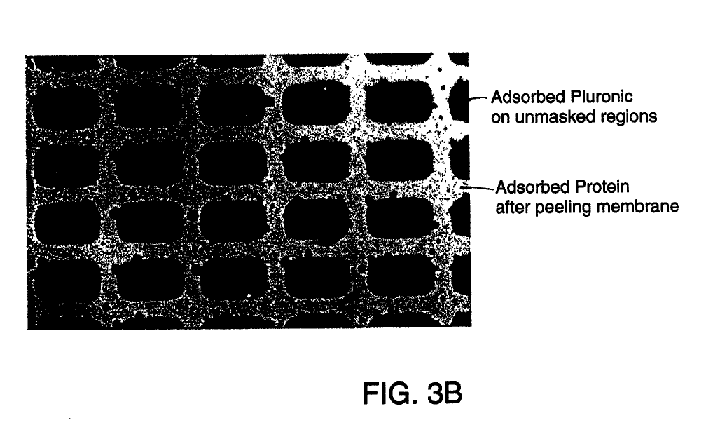

In yet another embodiment, patterns of adhesive and non-adhesive regions can be made using masks. By placing a thin membrane, e.g., of PDMS, onto a substrate, and then rinsing with surfactant, the adsorption of surfactant onto the masked regions is prevented. This method can be used to produce membranes with defined arrays of holes in the membranes such that the surfactant can be adsorbed onto the surface wherever a hole exists in the membrane (see FIG. 3).

Masks can also be used to pattern hydrophilicity on surfaces. By exposing a masked hydrophobic surface (e.g., bacteriological polystyrene petri dish) to a plasma etcher, the plasma reacts to the unmasked regions, rendering these regions hydrophilic. The surfactant then adsorbs only to the originally masked, hydrophobic regions.

In yet other embodiments of the present invention, the use of adhesive and non-adhesive agents can be combined with other techniques, such as photolithography and microfluidic patterning. Examples of such methods to control cell-biomaterial interactions include: (1) direct localization of cells through injection of a cell suspension into microfluidic channels, (2) indirect localization of cell adhesion by first patterning substrates with adhesive extracellular matrix molecules, or (3) indirect localization of cells by first patterning non-adhesive polyethylene oxide domains by simple adsorption of a commercial triblock polymer, Pluronic™ F108 on substrates. For example, as shown in the examples below, photolithographic and microfluidic patterning techniques are combined to direct localized coupling of PEO to a variety of biomaterial substrates by a simple adsorptive process. Using this technique, we demonstrate the ability to micropattern growth-competent 3T3 murine fibroblasts in 10% serum and retain cell-free domains for at least 2 weeks on polystyrene.

The methods of the present invention enable the co-culture of two or more cell types, e.g., hepatocytes and fibroblasts. Thus, these micropatterning tools provide methods to more accurately mimic the complexity of in vivo tissue architectures. Applications of these techniques include the control of and study of the role of the microenvironment around cells, e.g., hepatocytes, in vitro; cell and tissue engineering, tailoring biomaterial implants, and fundamental studies on signaling in cell-cell and cell-matrix interactions.

In addition, the methods of the present invention can be applied to hydrophilic surfaces, such as glass, by first rendering the (patterned) surface hydrophobic, e.g., using a methyl-terminated silane. The methods of the present invention can be combined with microfluidic patterning approaches to localize adsorption on model hydrophobic surfaces, e.g., polystyrene. Furthermore, other hydrophobic biomaterials can be similarly modified, e.g., PLGA (Poly(DL-lactide-co-glycolide) and polyimide.

As shown herein, the combination of previous methods of photolithographic patterning with fluidic delivery of cells allows both creation of isolated islands and patterning of multiple cell types with a limited number of ligands. Therefore, combination of these methods facilitates creation of more highly-structured multicellular culture environments.

The preferred binding agents are cytophilic, that is, adapted to promote cell attachment. Generally binding agents are those that would generally promote the binding, adherence, or adsorption of biological materials such as, for example, intact cells, fractionated cells, cellular organelles, proteins, lipids, polysaccharides, simple carbohydrates, complex carbohydrates, and/or nucleic acids. Molecular entities creating cytophilic surfaces are well known to those of ordinary skill in the art and include compounds that have functional groups that include hydrophobic groups or alkyl groups with charged moieties such as —COO⁻, —PO₃H— or 2-imidazolo groups, and include compounds or fragments of compounds such as antigens, antibodies, cell adhesion molecules, extracellular matrix molecules such as laminin, fibronectin, collagen, integrin, serum albumin, polygalactose, sialic acid, and various lectin binding sugars, synthetic peptides, carbohydrates and the like. Specifically the binding agents are those that selectively or preferentially bind, adhere or adsorb a specific type or types of biological material so as, for example, to identify or isolate the specific material from a mixture of materials. Specific binding materials include antibodies or fragments of antibodies and their antigens, cell surface receptors and their ligands, nucleic acid sequences and many others that are known to those of ordinary skill in the art. The choice of an appropriate binding agents depends on considerations of the biological material sought to be bound, the affinity of the binding required, availability, facility of ease, and cost. Such a choice is within the knowledge, ability and discretion of one of ordinary skill in the art.

The surface that is patterned can be any type of surface that useful for the desired application and that is known in the art. The term “surface” refers to the foundation upon which biomolecules may be immobilized, samples may be applied for analysis or biological assays may be carried out. The surface material may comprise any biological, non-biological, organic, or inorganic material, or a combination of any of these existing as particles, strands, precipitates, gels, sheets, tubing, spheres, containers, capillaries, pads, slices, films, slides, etc. The substrate may substantially planar, although it need not be according to certain embodiments.

Examples of useful materials include, but are not limited to, a variety of materials such as glass, quartz, silicon, alumina, polymers, gels, plastics, resins, carbon, metal, membranes, etc., other organic polymers including acrylonitrile-butadine-styrene copolymers, polysulfone, as well as bioerodable polymers including polyanhydrides or polylactic or polyglycolic acids, or from a combination of several types of materials such as a polymer blend, polymer coated glass, silicon oxide coated metal, etc. Other examples include polymers which contain a low intrinsic fluorescence emission, such as polycarbonate, polymethylmethacrylate (PMMA), and the like.

The surface material may be of any thickness depending on the intended use for the patterned material and can be readily selected by one of ordinary skill in the art. The surface includes one dimensional materials, e.g., wires, nanotubes, two dimensional materials, e.g., tissue culture plate or glass slide, and three dimensional surfaces, e.g., spheres, polymer constructs, etc. The surface may be corrugated, rugose, contoured, concave, convex or any combination of these. For example, it may be desirable to coat the region between the wells on a microtitre plate or other type of assay plate, with the surfactant. The surface may also be a prosthetic or implantable device on which it is desired to form a pattern of cells, proteins, or other biological materials. The word “surface” is used only for expository brevity and is not to be construed as limiting the scope or claims of the present invention to planar surfaces. Preferably the substrate is hydrophobic or can be rendered hydrophobic by known means. The shape of the surface can readily be selected by one of ordinary skill in the art based upon the desired application.

The patterned surfaces of the present invention may be used to create patterns of cells in which cells are isolated on islands to prevent cell to cell contact, in which different types of cells are specifically brought into contact or in which cells of one or more types are brought into a pattern which corresponds to the pattern or architecture found in natural tissue.

Such surfaces of patterned cells have a wide variety of applications which will be apparent to one of ordinary skill in the art and all such applications are intended to fall within the scope of this invention. Particularly preferred applications include but are not limited to use in bioreactors for the production of proteins or antibodies, especially by recombinant cells; use in tissue culture; use for the creation of artificial tissues for grafting or implantation; use in artificial organs such as artificial liver devices for providing liver function in cases of liver failure; and use for generating artificial tissues to adhere to the surfaces of prosthetic or implantable devices to prevent connective tissue encapsulation; non-fouling domains of diagnostics, drug delivery, in vitro microarrays.

The invention provides novel devices useful for adhering cells in specific and predetermined positions. Such devices are useful in a wide array of cellular biology applications, including cell culturing, recombinant protein production, cytometry, toxicology, cell screening, microinjection, immobilization of cells, influencing the state of differentiation of a cell including promoting differentiation, arresting differentiation or causing dedifferentiation. The devices of the invention also can be used to promote ordered cell-cell contact or to bring cells close to one another, but prevent such contact. The devices of the invention also are useful in the creation of artificial tissues for research or in vivo purposes and in connection with creating artificial organs such as artificial liver devices. The devices also are useful in connection with generating surfaces for prosthetic or implantable devices.

According to one aspect of the invention, a plate defining a surface with at least one cytophilic island is provided. The cytophilic island includes binding agents, e.g., proteins that are capable of binding the cells of interest. In one preferred embodiment, the device includes a plurality of such islands. These islands is isolated by a cytophobic region of a non-adhesive agent, which can be contiguous with the cytophilic island. Thus, islands on a plate are regions to which cells, proteins or other biological materials may be expected to adhere or bind. Islands of the foregoing type can take on virtually any shape when manufactured according to the methods of the invention. They also can be adapted to bind only selected cell types. Preferred islands are between 1 and 2,500 square microns, preferably between 1 and 500 square microns. In some applications, the islands can have an area of as little as between 1 and 100 square microns. Also according to the invention, the islands may have a lateral dimension of between 0.2 and 10 microns. The number of and distance between cytophilic islands can be altered by one of ordinary skill in the art depending on the desired use. For instance, if it is desirable to have some cell to cell interaction, the islands may be patterned to be close enough together for intercellular contact. Or alternatively, the distance between the islands can be enlarged by using a greater area of non-adhesive agent.

In one embodiment of the invention the cyotophilic regions are interconnected to form a circuit, e.g., to form a network of cells. This embodiment can be used for forming neuronal networks that function, e.g., as a microchip.

In yet another embodiment, the adhesive regions are aligned to form a parallel pattern of alternative adhesive and non-adhesive regions. When this type of patterned surface is contacted with cells or tissues, the cells align themselves along the lines of adhesive regions. This type of structure pattern could be applied to bandages and used in wound healing to accelerate tissue repair and minimize scarring. Such a pattern could also be used in repairing nerve damage. This type of structure would act, in essence, as a “smartbandage”.

The methods of the present invention are also useful in designing devices for use in diagnostic assays. In one embodiment, the surface of a plate could be patterned with islands where the islands are identical, e.g., containing a marker for a particular disease, and different patient samples are applied to each island. Thus, one could perform one assay to test a number of different samples for the same disease or presence of a marker. Alternatively, each island contains a different marker, e.g., different proteins, for different types of diseases. All the islands on this type of plate would the be contacted with one patient's sample.

The patterned surfaces of this invention are suitably used in an array format, i.e. where multiple test samples are analyzed substantially simultaneously on the substrate platform. As referred to herein, the term “array” indicates a plurality of analytical data points that can be identified and address by their location in two or three-dimensional space, where i.e. identify can be established by the data point physical address.

The adhesive regions of surfaces of the invention may be coated with a single biomolecule, with a random mixture of biomolecules or with a mixture of biomolecules wherein each unique biomolecule is located at a defined position so as to form an array. In one embodiment the surface is coated with a library of polypeptides or nucleic acids wherein each unique nucleic acid or amino acid sequence is located at a defined adhesive region on the surface.

The surfaces of the invention can be used for carrying out a variety of bioassays. Any type of assay wherein one component is immobilized may be carried out using the surfaces of the invention. Bioassays utilizing an immobilized component are well known in the art. Examples of assays utilizing an immobilized component include for example, immunoassays, analysis of protein-protein interactions, analysis of protein-nucleic acid interactions, analysis of nucleic acid-nucleic acid interactions, receptor binding assays, enzyme assays, phosphorylation assays, diagnostic assays for determination of disease state, genetic profiling for drug compatibility analysis, SNP detection, etc.

Identification of a nucleic acid sequence capable of binding to a biomolecule of interest could be achieved by immobilizing a library of nucleic acids onto the surface so that each unique nucleic acid was located at a defined position to form an array. The array would then be exposed to the biomolecule under conditions which favored binding of the biomolecule to the nucleic acids. Non-specifically binding biomolecules could be washed away using mild to stringent buffer conditions depending on the level of specificity of binding desired. The nucleic acid array would then be analysed to determine which nucleic acid sequences bound to the biomolecule. Preferably the biomolecules would carry a fluorescent tag for use in detection of the location of the bound nucleic acids.

Assays using an immobilized array of nucleic acid sequences may be used for determining the sequence of an unknown nucleic acid; single nucleotide polymorphism (SNP) analysis, analysis of gene expression patterns from a particular species, tissue, cell type, etc., gene identification; etc.

The patterned surfaces of the present invention are also useful in assays using immobilized polypeptides. For example, an immobilized array of peptides could be exposed to an antibody or receptor to determine which peptides are recognized by the antibody or receptor. Preferably the antibody or receptor carriers a tag, e.g., a fluorescent marker, for identification of the location of the bound peptides. Alternatively, an immobilized array of antibodies or receptors could be exposed to a polypeptide to determine which antibodies recognize the polypeptide.

An embodiment of the invention using patterned plates with a grid pattern, can be used in cytometry. For example, the numbers or ratios of different types of cells in a sample may be efficiently assayed by contacting the suspension with one of the plates of the present invention, allowing a period of time for the cells to bind, washing away any excess solution or unbound cells if necessary, and then identifying and counting the different cell types at the specific and predetermined locations of the biophilic islands. Because the size of the islands may be chosen such that no more than one cell may bind on any given island, because the locations and geometric pattern of the islands may be predetermined, and because the cells will remain at fixed locations during the cell counting, the patterned plates of the present invention provide for much greater efficiency and accuracy in cytometry. The methods and devices of the present invention can be readily applied to method of cytometry known in the art.

Merely by means of example, and without limiting the scope of the present invention, the following cytometric applications of the present invention are listed. The cytometry system provided by the present invention could be used in measuring the numbers and types of cells in blood, urine, cerebrospinal fluid, PAP smear, biopsy, ground water, sea water, riparian water, and reservoir water samples, and any other application in which there is a desire to determine the presence, number or relative frequency of one or more types of cells in a large sample of cells.

In another aspect of the present invention, a method of assaying the effects of various treatments and compounds on individual cells is provided. In particular, the invention provides the capability to assay the effects of various treatments or compounds on each of a great many individual cells plated at high density but separated from each other and at fixed locations on the plate. In this embodiment of the invention, many cells are applied in suspension to the plates of the present invention.

Once the suspension of cells has been applied to the plate, a period of time is allowed to elapse in order to allow the cells to bind to the islands. Excess fluid including unbound cells may be washed away. The cells may then be subjected to a treatment or exposed to a compound in situ on the plate or, in some situations, the cells may be pre-treated before being introduced to the plate for binding. The effects of the treatment or compound on each cell may then be individually assayed in a manner appropriate to the cell type and the treatment or compound being studied. For example, the effects of treatments or compounds potentially capable of affecting cell morphology may be assayed by standard light or electron microscopy. Alternatively, the effects of treatments or compounds potentially affecting the expression of cell surface proteins may be assayed by exposing the cells to either fluorescently labeled ligands of the proteins or antibodies to the proteins and then measuring the fluorescent emissions associated with each cell on the plate. As another example, the effects of treatments or compounds which potentially alter the pH or levels of various ions within cells may be assayed using various dyes which change in color at determined pH values or in the presence of particular ions. The use of such dyes is well known in the art. For cells which have been transformed or transfected with a genetic marker, such as the β-galactosidase, alkaline phosphatase, or luciferase genes, the effects of treatments or compounds may be assessed by assays for expression of that marker and, in particular, the marker may be chosen so as to cause spectrophotometrically assayable changes associated with its expression.

In certain embodiments, the assay is spectrophotometric and automated. In these embodiments, the treatment or compound potentially causes a change in the spectrophotometric emissions, reflection or absorption of the cells. A detector unit, as described above, may be employed. Because of the small distances between individual isolated cells permitted by the present invention, detectors employing fiber optics are particularly preferred. Such sources of electromagnetic radiation and such detectors for electromagnetic transmission, reflection or emission are known in the applicable art and are readily adaptable for use with the invention disclosed herein.

In certain embodiments, a suspension of cells is applied to one of the plates of the present invention in which the binding agent is chosen so as to selectively or preferentially bind a certain type or types of cells. The cells are subjected to a treatment or exposed to a compound which will potentially cause a change in the electromagnetic emission, reflection or transmission characteristics of the cells and an automated detector unit records the emission, reflection or transmission characteristics of each cell individually by assaying electromagnetic emission, reflection or transmission at points corresponding to each island on the plate.

When an automated detector unit is used, a plate which has not been exposed to any cells may be used as a control before testing the experimental plate to provide reference values to exclude from the results islands on the experimental plates which have been exposed to cells but which have not bound cells.

In another embodiment, plates upon which cells have been allowed to bind are assayed prior to any potentially effective treatment or compound and then treated or exposed. As the cells maintain their individual positions on the plates, a second assay may be performed to detect changes in the assay results on a cell-by-cell basis after treatment or exposure. Such a two-step assay is particularly appropriate for treatments or compounds which potentially cause cell toxicity or disrupt binding.

The above described embodiments, employing the methods of the present invention which allow for plating individual cells at high density but with little or no overlap or contact of cells, can be employed for high through-put tests of potentially useful treatments including radiation and pharmacological or toxicological compounds. In particular, the present invention provides assays which allow assays both as to qualitative and quantitative changes in individual cells and quantitative assays as to percentages of cells affected by any given treatment or compound.

In a different embodiment, the present invention provides means for identifying individual cells which have been successfully transformed or transfected with recombinant DNA technology. A culture of cells exposed to transforming or transfecting vectors, including plasmids, phasmids, cosmids, retroviruses and various homologous recombination or integration elements, may be plated on the plates of the present invention to separate the cells and cause them to bind individually at the locations of the islands on the plate. Individual cells which have been transformed or transfected may then be identified by the methods described above or other methods well known to those of ordinary skill in the art. Particularly simple, given the disclosures herein, is the identification of individual cells transformed or transfected with a vector including a marker locus which causes a spectrophotometrically detectable change in a cell's function, metabolism, gene expression or morphology. Marker loci may also be included which cause cells to exhibit a sensitivity or resistance to a particular treatment or compound. Cells transformed or transfected by such vectors may be first selected on the basis of the appropriate sensitivity or resistance and then plated as individual cells and further selected or characterized by the methods and employing the plates described herein. In particular, selection may be employed prior to plating on the plates of the present invention to isolate transformed or transfected cells and then the cells may be assayed in situ using the presently disclosed materials and methods to identify and isolate cells with, for example, particularly high or low expression of the characteristic to which the transformation or transfection was directed.

In a different embodiment, the present invention provides materials and methods for retrieving individual cells which are bound to the plates of the present invention. That is, the present invention provides for materials and methods for isolating and manipulating particular individual cells which are present on a plate containing a great multiplicity of cells separated one from another by only a few microns.

Given the disclosures of the present invention for isolating individual cells on islands at predetermined positions on one of the disclosed plates, the design and production of a cell retrieval unit is within the ability of one of ordinary skill in the applicable art. Absent the present disclosure, retrieval of a particular individual cell from amongst a high density plate of a great many cells would be an arduous and difficult task. The binding of individual cells to particularly defmed positions on the plates of the present invention, however, provides for a method of such retrieval. Such a cell retrieval system may be employed, for example, to retrieve transformed or transfected cells, potentially cancerous cells in a PAP smear or biopsy, or fertilized eggs adhered to the patterned plates of the present invention.

In another aspect of the present invention, patterned plates and a method are provided for immobilizing cells for microinjection. As is known in the art, microinjection of, for example, dyes, proteins, and DNA or RNA sequences, is made more difficult when the cells to be microinjected are not immobilized on a substrate and/or localized at specific and predetermined positions. By providing the patterned plates and methods disclosed herein, the present invention greatly simplifies the microinjection process. Thus, in light of the present disclosure, patterned plates with biophilic islands which can bind a given type or types of cells can be produced and the type or types of cells can be bound individually to specific and predetermined locations on the plates. Cell types which may be sought to be bound include bacterial cells such as Escherichia and Pseudomonas species; mammalian cells such as chinese hamster ovary (CHO), baby hamster kidney (BHK), hepatocytes, COS, human fibroblast, hematopoietic stem cells, and hybridoma cell lines; yeast; fungi; and cell lines useful for expression systems such as yeast or Xenopus laevis oocytes. The listing above is by no means intended to be exhaustive but is merely exemplary of the sorts of cells which may be immobilized to specific and predetermined positions for microinjection. Subsequent to microinjection, the cells may be assayed for functional expression or transformation on the plates of the present invention with the detectors described herein and, if desired, individually retrieved with the retrieval system disclosed herein.

In another aspect of the present invention, materials and methods are provided which allow for the immobilization of oocytes at specific and predetermined positions for in vitro fertilization techniques. That is, the patterned plates of the present invention allow for immobilization of oocytes, including human oocytes, at specific and predetermined positions. These immobilized oocytes may then be contacted in situ on the plates with a solution including sperm cells potentially capable of fertilizing the oocytes. The fertilized oocytes, or zygotes, may then be conveniently identified because of their fixed positions on the plates of the present invention and individually retrieved for implantation or storage by standard methods or the methods disclosed herein. One of ordinary skill in the art can readily select the appropriate binding agents for immobilizing the oocytes/zygotes based on their knowledge of the art, e.g., including moieties, including antibodies, which specifically bind the oocytes/zygotes involved in the in vitro fertilization process. Subsequent to exposure to the sperm solution, the cells may be assayed for successful fertilization on the plates of the present invention with the detectors described herein and, if desired, individually retrieved with the retrieval system disclosed herein.

In another aspect of the present invention, patterned plates are provided which may be used to bind or adsorb proteins in specific and predetermined patterns. As is known to those of ordinary skill in the art, phenomena associated with the adsorption of proteins to solid synthetic materials are important in many areas of biotechnology including, for example, production, storage and delivery of pharmaceutical proteins, purification of proteins by chromatography, design of biosensors and prosthetic devices, and production of supports for attached tissue culture.

In a different embodiment of the present invention, the patterned plates provided herein may be used to produce plates with cells growing in desired patterns and to control the growth, proliferation, differentiation, orientation and/or spreading of certain classes of cells.

As in the previously described embodiments of the present invention, depending upon the intended use, an enormous variety of patterns may be produced and a multiplicity of stamps and/or a multiplicity of binding agents may be employed to create patterns of one or more types of cells.

In another embodiment particular different types of cells may be brought together on the same plate. For example, it may be desired to plate a percentage of one type of cells, e.g., hepatocytes, on the plate to convert potentially procarcinogenic compounds into carcinogenic compounds and to assay the effects on other types of cells, e.g., nonhepatocyte cells, on the same plate.

The present invention provides a simple, chemically-generic tool for patterning non-adhesive domains, e.g., by using PEO. This tool for customizing cell culture environments by specifying non-adhesive domains is useful for many different cell types rather than specifying adhesive domains with specific integrin-binding ECM molecules. Due to the use of surface hydrophobicity rather than chemistry (gold, silicon) to immobilize PEO, this technique is useful for a wide range of conventional biomaterials that have carbon-backbones. Indeed, Patel et al recently described the use of microfluidics to render a PLGA template adhesive via modification with adhesive peptides [43]. We propose a similar approach for PEO immobilization. This level of flexibility broaden the utility of this tool to other fundamental cell and tissue engineering applications.

The methods described herein enable the customization of cell culture environments for cell and tissue and engineering. The combination of microfluidic and photolithographic patterning as well as simple adsorption of adhesive (ECM) and non-adhesive (PEO) species can be extended to novel applications such as: modification of the PPO Pluronic core with adhesive peptides to create surfaces with well-defined adhesivity [25], use of degradable triblocks (PEO-PLGA-PEO) to dynamically modulate adhesivity [58], and novel substrates such as PEO lipid bilayers [59] and biomaterials (PLGA)[43]. Furthermore, the patterning modes utilized can be used in microcontact printing of proteins [60-62] and microfluidics with polymer or hydrogel actuation [28,63].

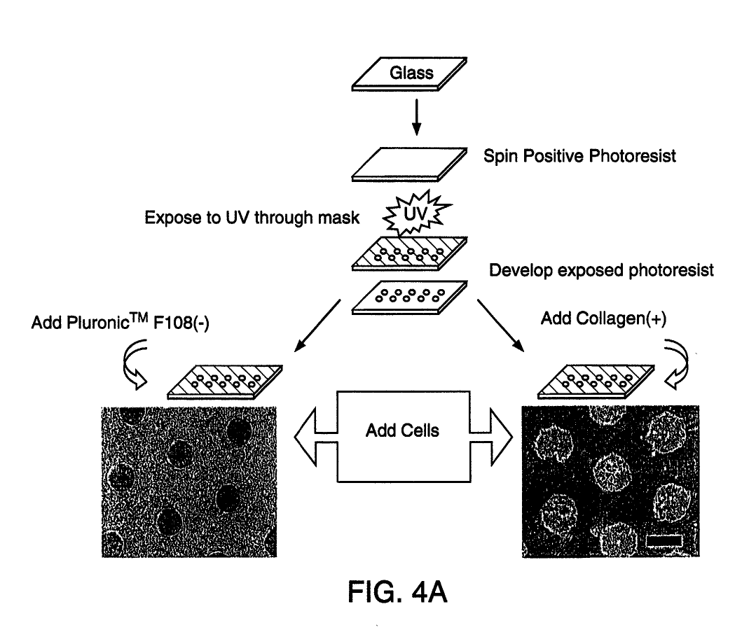

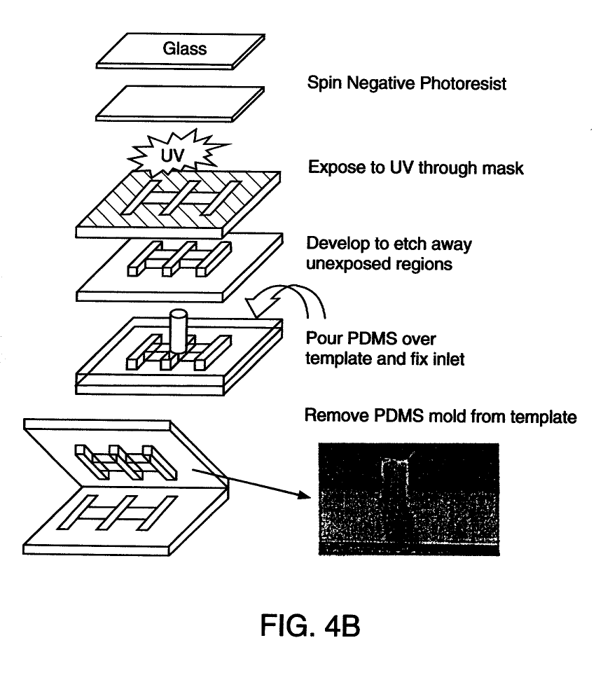

Certain embodiments of the methods of the present invention provide a combination of photolithographic and microfluidic patterning schemes that are utilized to localize both adhesive and non- adhesive moieties. FIG. 4A schematically depicts direct photolithographic patterning of glass substrates with an extracellular matrix protein (collagen I) that is adhesive for many cell types, or PEO polymers that are non-adhesive for both proteins and cells. Both primary cells (primary rat hepatocytes) and immortal cell lines (3T3 fibroblasts) were patterned using these surface modifications.

In contrast, FIG. 4B depicts a patterning scheme for extracellular matrix, PEO, and direct cell localization via a fluidic delivery system constructed from polydimethylsiloxane (PDMS). Fluidic channel networks were molded by casting PDMS on a pre-fabricated template. Upon curing, PDMS is well known to form a self-sealing elastomer. When placed in contact with a rigid substrate, the channels allow localized access to the underlying substrate; therefore, perfusion of channels with adhesive or non adhesive chemical species which can spontaneously adsorb or be covalently coupled to the surface facilitated localized immobilization on the underlying, chemically-uniform substrate. Similarly, mammalian cells can be directly injected into these channels and therefore allowed to attach only in specified regions of the underlying substrate.

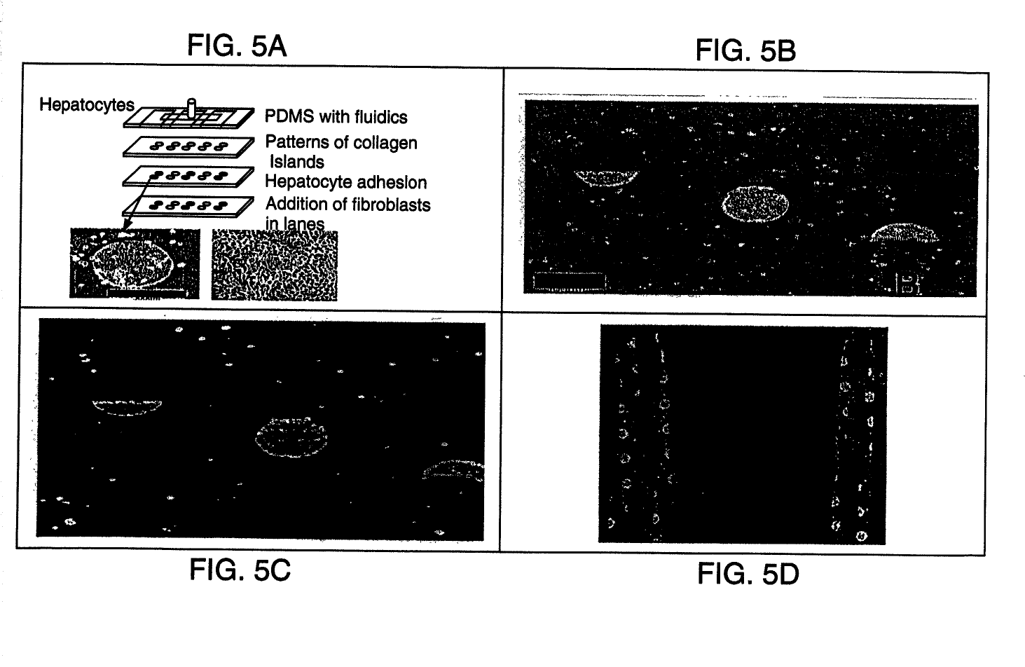

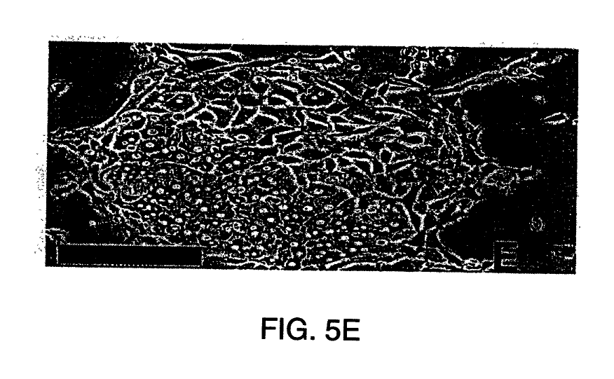

The results achieved by combining photolithographic patterning and microfluidic patterning to combine the advantages of each technique individually are shown below. In particular, photolithographic patterning allowed a simple method to produce isolated structures (i.e. islands) with varying periodicity and size and shape. In contrast, microfluidic patterning allows patterning to be achieved on a variety of materials that are not amenable to conventional photolithographic methods-polystyrene, teflon, poly-lactide-co-glycolide, etc. Furthermore, microfluidic patterning has the theoretical advantage that a number of networks can be accessed separately- i.e. one network can be perfused with one cell type while the adjacent network can be used to localize a distinct cell type or chemical species. In FIG. 5, we demonstrated that fluidic localization of cells on a photolithographically-patterned substrate (rather than a chemically uniform one) can be used as a simple method to create many repeated isolated structures in a localized region of a substrate.

FIG. 5A shows a method of the present invention for injection of primary cells into a microfluidic network placed upon a previously collagen-patterned surface, thereby localizing domains of micropatterned islands on the underlying surface. Indeed, when this approach was combined with a second cell type, we simultaneously achieved sub-domains of distinct structural characteristics (i.e. co-culture versus cultures of one cell type) on a single substrate. In addition, this technique offers the potential to micropattern two different cell types simultaneously on the same adhesive ligand as seen in FIG. 5E (e.g., collagen) rather than using 2 distinct surface chemistries that select for cell adhesion by binding to distinct cell adhesion molecules. Conversely, cell populations with similar surface receptor populations can be localized in various sub-domains of a single substrate by physical separation of individual fluidic networks.

The methods described in the examples below which combine microfluidic and photolithographic patterning have a number of particularly notable aspects. For example, photolithographic patterning of silane surface chemistries, proteins, or adhesive ligands has been reported [6,16, 32, 42]. These techniques result in two distinct surface properties which can then be used to specify coil adhesion; however, cells are typically plated over the entire patterned surface. Thus, distinct sub-domains cannot be achieved. Similarly, microcontact printing can be utilized to generate patterns of adhesive species on gold thin films using self-assembled monolayers of alkane-terminated thiols, which adsorb adhesive proteins. This technique has recently been modified to microcontact printing of proteins directly; therefore, multiple protein patterns in distinct sub-domains can be theoretically achieved. In order to pattern distinct cell types, however, cell-specific adhesive ligands must be utilized to ‘sort’ cells from solution rather than employing microfluidics for spatial localization as seen in the current study [64]. Practically, this limits the number of distinct cell types that can be simultaneously sorted to those which have at least one distinct adhesion receptor even though the full complement of cell surface receptors are rarely known for every cell type in culture.

Similarly, microfluidic patterning has been previously reported for localization of adhesive peptides and proteins [10,11,43] as well as direct localization of cells [12,17]. A well-recognized disadvantage of microfluidic patterning was the necessity for the resulting cellular pattern to be spatially continuous. Whitesides and coworkers have reported a 3-dimensional adaptation of soft lithographic microfluidic methods that allowed localization of cells and adhesive molecules in isolated islands [12]. However, this method has its disadvantages due to a limitation on perfusion pressure and the frequency with which island can be repeated will be limited by both the lower limitation on well size, and the inter-well adhesive portion of the PDMS. In contrast, the limitations on island size and spatial frequency using the methods of combining photolithography and microfluidics in the present invention are dictated by the photolithographic process utilized to fabricate the underlying patterned substrate (˜0.1 μm), or practically by the size of a single cell (˜10-20 μm diameter).

Another aspect of previous studies to use microfluidics as a vehicle to localize cell delivery to a substrate, is the limited application of these techniques to cell types that will attach and spread in a relatively anoxic environment. Although PDMS is known to be relatively oxygen permeable, some primary cells require a relatively high oxygen flux (oxygen uptake rate of 0.3-0.9 nm/s/10⁶ cells for primary hepatocytes [44]) to drive the attachment and spreading process that can not be achieved by passive diffusion of oxygen from room air through PDMS [36]. In the method of the present invention, we increased the dissolved oxygen in the cell suspension by pre-equilibrating with 90% O₂/10% CO₂ mixture, thus providing sufficient oxygen for selective adhesion and spreading of primary hepatocytes. Alternatively, we have achieved similar results by utilizing channels with a large perfusion volume, thereby increasing the amount of dissolved oxygen available for cell metabolism during the 2 hours required for adhesion.

The method of the present invention enable a different surface chemistry than that typically utilized in microfluidic patterning. Others have relied on the use of substrates or ligands that an adhesive for cells, whereas here we demonstrate that microfiudics (in conjunction with photolithography) can also be utilized to specify non-adhesive domains. Indeed, patterning by deterring adhesion, can be generalized more readily across cell types and species sources as it does not rely on the presence of specific cell surface adhesion molecules.

The Examples below show that the non-adhesive regions, i.e., coated with surfactant, of the surface is able to resist protein adsorption from culture media and therefore provides a useful in vitro tool for patterning non-adhesive domains. These results, together with in vivo data on exposure to whole human plasma suggests that the methods of the present invention will provide a valuable in vivo tool to control early host-biomaterial interactions [18]. In comparison to other previously described techniques for patterning polyethylene oxide domains, the current method of localizing adsorption of triblock polymers via microfluidics or photolithography offers a simple, versatile alternative.

The present invention is further illustrated by the following Examples. The Examples are provided to aid in the understanding of the invention and are not construed as a limitation thereof.

All examples are carried out using standard techniques, which are well known and routine to those of skill in the art, except where otherwise described in detail. Routine techniques of the following examples can be carried out as described in standard laboratory manuals.

### General Comments

The following methods were used in the following Examples.

Microfabrication tools were utilized to achieve patterning of adhesive (collagen I) and non-adhesive (PEO) moieties in two distinct modes and combinations thereof: 1. direct photolithographic patterning and 2 microfluidic patterning using an elastomer mold. Direct photolithographic patterning is achieved by coating substrates with a light-sensitive polymer (photoresist) followed by exposure, development and chemical modification of selected regions with adhesive or non-adhesive species. In contrast, microfluidic patterning is achieved by microfabricating a textured template, subsequent casting of an elastomer mold on this template and use of the resulting elastomer channel network to localize delivery of adhesive or non-adhesive species to the surface of a substrate. Primary cells (purified rat hepatocytes) and cell lines are cultured alone or together (co-cultures) in various spatial arrangements. Finally, surface modification of various substrates (glass, polystyrene, PDMS) is achieved either by adsorption or covalent grafting to an immobilized silane. Photolithographic and microfabrication methods are schematized in FIG. 1.

### Photolithographic Patterning

Detailed procedures for photolithographic patterning of substrates and subsequent modification were previously described [16] and are depicted schematically in FIG. 1A. Briefly, 2″ diameter×0.02″ borosilicate wafers (Erie Scientific; Portsmouth, N.H.) were spin-coated with positive photoresist (S1813, Shipley). Wafers were baked and then exposed to ultraviolet light in a Bottom Side Mask Aligner (Karl Suss, Waterbury Center, Vt.) through emulsion masks of the desired dimensions. We utilize emulsion mask as an inexpensive, readily available alternative to chrome/quartz masks. Patterns are drawn in Corel Draw 9.0* and printed using a commercial Linotronic-Hercules 3300 dpi high-resolution line printer. Exposed photoresist was then developed (MF-319 developer, Microchem Corporation, Waltham, Mass.: water, 1:1), baked, and finally cleaned by exposure to oxygen plasma for 10-15 min.

Some patterned substrates were then modified by covalent coupling of collagen I using experimental techniques previously described in detail. Briefly, silane immobilization onto exposed glass was performed by immersion into 2% v/v solution of 3-[(2-aminoethyl)amino] propyltrimethoxysilane (AS, Huls America, Piscataway, N.J.) in water, 2.5% v/v glutaraldehyde in phosphate-buffered saline (PBS, pH 7.4), and a 1:1 solution of 1 mg/mL collagen I (preparation from rat tail tendons described in detail elsewhere, 17): DI water, pH 5.0 for 30 min at 37° C. Alternatively, collagen I was adsorbed onto patterned substrates by incubation with 0.6 mg/mL collagen I in water for 1 h at 37° C. Discs were finally sonicated in acetone for 3 min to remove residual photoresist (Bransonic) and create a micropatterned substrate of collagen/glass.

### Microfluidic Patterning

Techniques for microfluidic patterning were adapted from Folch et al [7]. Briefly, a high-aspect ratio photoresist (SU-8, Microchem Corporation, Waltham, Mass.) (25 um thick) was spun on silicon wafers (Virginia Semiconductor, Fredricksburg, Va.) and exposed to ultraviolet light through an emulsion mask as described above and developed according to manufacturer specifications. This template was used as a mold for casting polydimethylsiloxane (PDMS; Sylgard 184, Dow Corning). PDMS was prepared, degased under low vacuum, poured over the SU-8 template, and cured at 65° C. for 2 hours. Before curing, we fixed a small piece of tubing to the PDMS mold to serve as an inlet (MastexFlex, Ill.). The PDMS mold is subsequently removed from the SU-8 template and used as a network of microchannels when superimposed upon a rigid substrate. PDMS forms an aqueous seal with rigid substrates and can therefore serve as a vehicle for the localized delivery of adhesive or non-adhesive factors or cells suspended in media.

### Combined Photolithographic and Microfluidic Patterning

For some experiments, photolithographically- patterned glass substrates were utilized for subsequent microfluidic cell deposition. Glass wafers with collagen-immobilized domains were utilized in conjunction with PDMS fluidic channels fabricated as described above. Recall, previously that cells were deposited uniformly by direct injection into fluidic channels; however, in this case we achieved 2 distinct modes of spatial localization: (1) through patterning of ‘adhesive’ domains on underlying substrate, and (2) through localization of cell delivery to selected portion of the substrate. We have previously reported that hepatocyte attachment is an oxygen-dependent process [36]; therefore, in order to achieve selective adhesion of hepatocytes to patterned domains within the fluidic channels we either: (1) pre-oxygenated the hepatocyte solution by bubbling with 90%O₂, 10% CO2, or (2) fabricated relatively deep fluidic structures (−3 mm) which contained greater amounts of dissolved oxygen due to the relatively large fluid volume.

### PEO Coupling

Pluronic™ F108 was selected from a family of triblock polymers that are commercially available. (BASF, #F-108). This class of polymers have polypropylene centers with polyethyelene oxide side chains with the following proportions (PEO)₁₂₉-(PPO)₅₆-(PEO)₁₂₉ and a molecular weight of 14,600 g/mole. The polypropylene domain adsorbs quasi-irreversibly to hydrophobic surfaces, creating a surface coating of PEO chains, thus surfaces that are hydrophobic can be modified with PEO regardless of their chemical composition [18]. While chain length of the PEO domain can vary, Li et al have previously reported that PIuronic™ F108 was most suitable for deterring protein adsorption within a group of Pluronics with varying PPO and PEO domians. Solutions of 1 or 4% w/w Pluronic™ F108 in water were prepared, injected into microfluidic networks that were opposed to a hydrophobic surface and allowed to adsorb for 24 h at room temperature. Alternatively, hydrophilic surfaces such as glass were photolithographically patterned, rendered hydrophobic by modification with 5% dimethyltrichlorosilane in chlorobenzene, then stripped of photoresist in acetone, and finally incubated with Pluronic™ P108.

### Cell Culture

### Hepatocytes

Hepatocytes were isolated from 2- to 3-month-old adult female Lewis rats (Charles River Laboratories, Wilmington, Mass.) weighing 180 200 g, by a modified procedure of Seglen [37]. Detailed procedures for isolation and purification of hepatocytes were previously described by Dunn et al [38]. Routinely, 200-300 million cells were isolated with viability between 85% and 95%, as judged by trypan blue exclusion. Nonparenchymal cells, as judged by their size (<10 μm in diameter) and morphology (nonpolygonal or stellate), were less than 1%. Culture medium was Dulbecco's modified eagle's medium (DMEM, Gibco) supplemented with 10% fetal bovine serum (FBS, Sigma, St. Louis, Mo.), 0.5 U/mL insulin, 7 ng/mL glucagon, 20 ng/mL epidermal growth factor, 7.5 μg/mL hydrocortisone, 200 U/mL penicillin, and 200 μg/mL streptomycin. Serum-free culture medium was identical except for the exclusion of FBS.

### NIH 3T3-J2 Fibroblast Culture

NIH 3T3-J2 cells were the gift of Howard Green, Harvard Medical School. Cells grown to Preconfluence were passaged by trypsinization in 0.01% trypsin (ICN Biomedicals, Costa. Mesa, Calif.)/0.01% EDTA (Boehringer Mannheim, Indianapolis, Ind.) solution in PBS for 5 min, diluted, and then inoculated into a fresh tissue culture flask. Cells were passaged at pre-confluency no more than 10 times. Cells were cultured in 175 cm² flasks (Fisher, Springfield, N.J.) at 10% CO₂, balance moist air. Culture medium consisted of DMEM (Gibco, Grand Island, N.Y.) with high glucose, supplemented with 10% bovine calf serum (BCS, JRH Biosciences, Lenexa, Kans.) and 200 U/mL, penicillin and 200 μg/mL. streptomycin.

### Cell Adhesion Assays

In order to quantify cell adhesion to Pluronic™ F108-treated surfaces and explore the potential to combine Pluronic™ F108-treated surfaces with specific ECM molecules, cell adhesion to various substrates was determined by light microscopy and image analysis. 125,000 hepatocytes or fibroblasts were initially plated on various substrates in the absence of serum to determine their propensity for mediating cell adhesion. Substrates included: polystyrene (tissue-culture treated) controls, polystyrene+Pluronic™ F108, polystyrene+collagen, polystyrene+Pluronic™ F108+collagen. After 24 h, unattached cells were removed, plates ware washed with fresh medium and imaged by phase contrast microscopy. We quantified adhesion using Metamorph Image Analysis software in 2-16 fields per condition.

### Microscopy

Specimens were observed and recorded using a Nikon Diaphot microscope equipped with a SPOT digital camera (SPOT Diagnostic Equipment, Software Version 2.2, Sterling Heights, Mich.), and MetaMorph Image Analysis System (Universal Imaging, Westchester, Pa.) for digital image acquisition. Fluorescent labels CMFDA (chloromethylfluorescein diacetate, C-2925, Molecular Probes) and CMTMR (chloromethylbenzoylaminotetramethyl rhodamine, C-2927) were utilized to track cells fluorescently. Cells were loaded by incubation in 25 μM dye in media for 45 min, rinsed, and incubated for 30 min prior to a final rinse. Cells were observed by fluorescence microscopy with ex/em: 492/517 and 541/565 nm.

### Statistics and Data Analysis

Experiments were repeated two to three times with duplicate or triplicate culture plates for each condition. One representative experiment is presented where the same trends were seen in multiple trials. Error bars represent standard error of the mean. Statistical significance was determined using one-way ANOVA (analysis of variance) on Statview with Fisher's PLSD Post-Hoc analysis with p<0.05.

### EXAMPLE 1

FIG. 5A provides a schematic depiction of the method to localize hepatocytes through fluidic network on glass substrate patterned with collagen I islands. Hepatocyte suspension is pre-oxygenated by bubbling with 90%O₂/10%CO₂ to supply oxygen for hepatocyte attachment and spreading [36]. Hepatocytes were injected, allowed to attach for 2 h, and the PDMS network was removed. FIGS. 5B and C show hepatocytes that were patterned using this technique by phase contrast microscopy and fluorescence respectively. In this case, the perfused PDMS channel was placed horizontally over a pre-patterned array of 500 μm collagen islands. Therefore, hepatocytes have full access to central islands but only partial access to peripheral islands. This is seen in B and C by the presence of both circular islands as well as semi-circle patterns of hepatocyte adhesion.

Addition of a second cell population by injection into the fluidic network would form individual repeating domains of micropatterned co-cultures. Two such domains are visualized fluorescently in FIGS. 5D. Hepatocytes, fluorescently-labeled green are visualized as repeating islands in rectangular domains whereas 3T3 fibroblasts that were subsequently seeded through the PDMS network are visualized in red. Removal of the PDMS network yields 2 distinct micropatterned co-culture subdomains. Thus, the ability to form hierarchical architectures can be realized with the ability to specify tissue structure on the cellular length scale (˜10-20 μm) as well as tissue length scales (˜1 mm).

Finally, this technique was utilized to create a unique structure that, to our knowledge, is not achievable by other means. FIG. 5E depicts an isolated collagen I island on a glass substrate. One island was partially exposed to hepatocytes as seen in 2B. Removal of the PDMS mold after hepatocyte adhesion and spreading revealed the remainder of the collagen-coated island. Therefore, upon application of a second cell suspension, fibroblasts attached and spread to newly exposed sites, creating a ‘hybrid’ island on the same underlying extracellular matrix protein. Thus, the same ligand was utilized to pattern two distinct cell types in a spatially contiguous structure. As the field of tissue engineering advances, the ability to customize tissue architecture with these techniques may provide valuable insight on the structure/function relationship in complex multicellular tissues.

### EXAMPLE 2

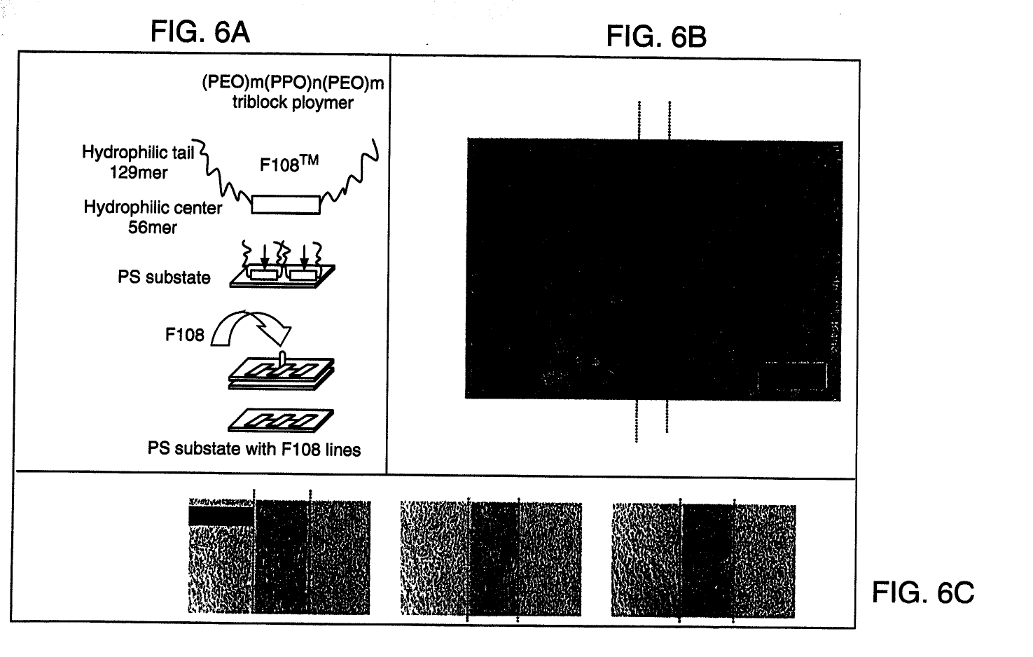

Photolithographic and microfluidic modalities were also utilized to localize PEO on biomaterial substrates. Previously, patterns of PEO have been achieved through self-assembled monolayers on gold[26], photopolymerization of interpenetrated networks (poly (acrylamide-co-ethylene glycol)) [39], or silane-based coupling of PEO to Si-based materials [32]. Neff et al used this technique to passivate polystyrene and then specifically grafted adhesive peptides such as RGD to study cell adhesion on a non-adhesive background [25, 33]. We localized F108 by both microfludic and photolithographic means. FIG. 6A schematically depicts the process by which F108 will adsorb, quasi-irreversibly to hydrophobic surfaces [18]. The length of PEO chains has been evaluated previously by Neff et al. F108 is the preferred analogue of the triblock copolymer. In FIG. 6B we demonstrated that PEO can be localized using PDMS microfluidics as described in FIG. 4B. 50 micron lanes of PEO were deposited on tissue-culture polystyrene by injection of a 4% F108 solution in water at room temperature and incubation for 24 h. Fluorescently-labeled murine 3T3 fibroblasts were subsequently seeded in the presence of 10% serum and attachment was subsequently deterred from PEO regions.

### EXAMPLE 3

Previous studies on this family of triblock copolymers has highlighted the potential for serum proteins to elute F108 from the surface. In order to evaluate the potential for F108 desorption in a conventional cell culture environment, we followed patterned fibroblast cultures in the presence of 10% serum over time. FIG. 6C demonstrates that the integrity of the cellular pattern was preserved for 2 weeks. Thus, any desorption of F108 from the surface was not sufficient to promote cell adhesion in previously non-adhesive regions. Indeed, this effect seemed to be cell-type dependent. For example, primary cells (hepatocytes) encroached onto non-adhesive areas much more rapidly (˜days rather than weeks) indicating that active cell processes such as ECM production or phagocytosis of F108 may alter its efficacy to deter cell adhesion.

### EXAMPLE 4

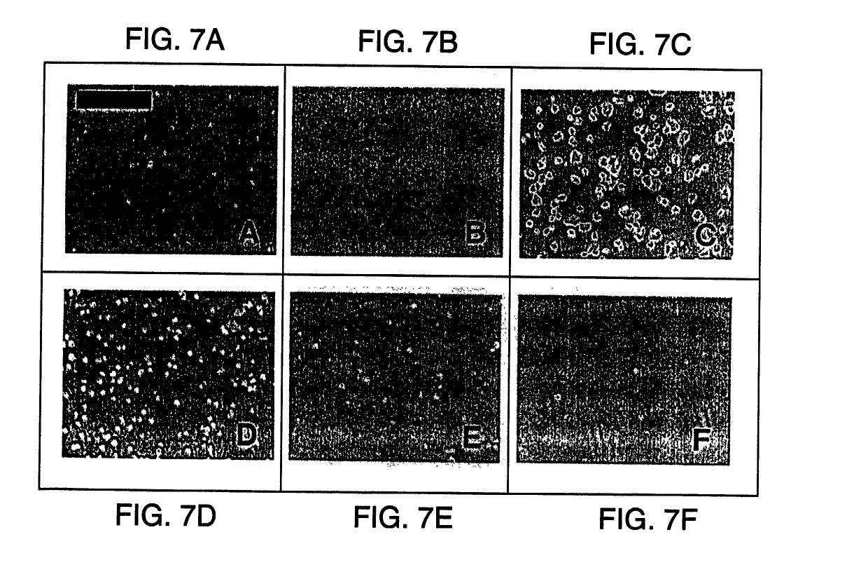

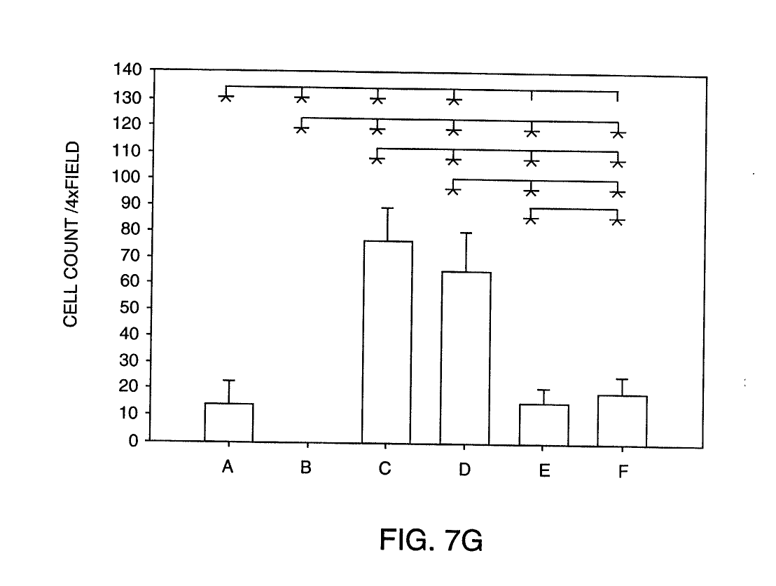

Qualitative variations in cell adhesion to F108-treated surfaces led us to characterize cell adhesion more quantitatively. In addition, since many cell culture strategies require the presence of serum or extracellular matrix proteins, we also characterized cell adhesion on ECM-coated, F108-treated surfaces. Specifically, we probed a model cell line (murine 3T3 fibroblasts) and a model primary cell (rat hepatocytes) under various conditions (I-100 μg/mL collagen I coating). FIG. 7 depicts the result of our studies with primary hepatocytes. Cell adhesion on polystyrene in the absence of serum was minimal (˜10 cells per field) and was completely eliminated by treatment with F108. In contrast, pre-treatment of polystyrene with 100 ug/ml of collagen for 1 h markedly increased cell adhesion (FIG. 7C, ˜75 cells per field). Finally, we explored the possibility that F108-passivated surfaces could be rendered adhesive by exposure to high concentrations of adhesive proteins (such as may occur physiologically in serum). Our data indicated that exposure to 1-10 μg/mL collagen had a modest effect on cell adhesion though substrates were no more adhesive than control polystyrene. In contrast, treatment of F108-passivated surfaces with 100 μg/mL collagen I effectively rendered the surface adhesive (FIG. 7D). Similar trends were observed with fibroblasts (data not shown).

### EXAMPLE 5

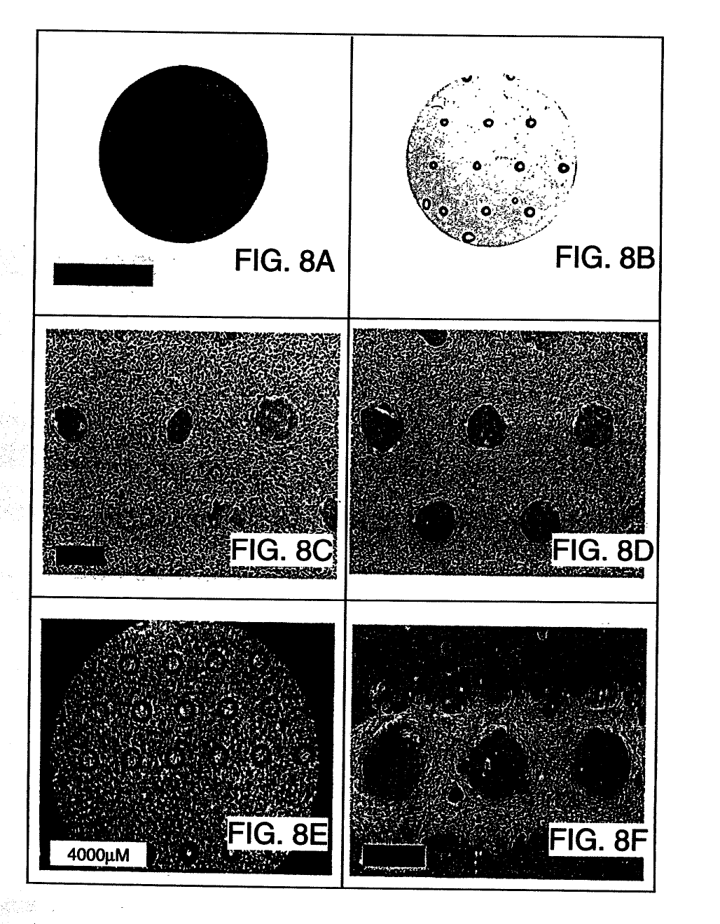

The localization of PEO through microfluidic channels in contact with hydrophobic surfaces, though useful and chemically-generic, could not be utilized on a common experimental substrate- glass. Indeed, hydrophilic surfaces cannot be directly modified using this adsorptive process. In order to demonstrate the feasibility of using this technique in conjunction with F108 coupling, borosilicate (glass) wafers were first rendered hydrophobic by coupling of a methyl-terminated silane. FIGS. 8A and 8B depict a model photoresist pattern utilized to demonstrate the change in contact angle resulting from methyl-termination. FIG. 8A is a fluorescent micrograph of autofluorescent photoresist on glass. Methyl-termination of exposed (black in A) glass, followed by removal of photoresist, results in increased contact angle of water droplets placed on the surface. FIG. 8B depicts the array of water droplets that result from such a surface modification-essentially encircling each droplet with a hydrophobic, methyl-terminated ring. Subsequent adsorption of PEO to modified glass then rendered the glass non-adhesive (data not shown).

### EXAMPLE 6

To farther broaden the utility of PEO coupling, we created PEO islands rather than continuous networks that are easily achieved using microfluidic networks. In order to achieve this, we photolithographicahy patterned islands of 500 um diameter. These regions were modified, as described above, by coupling to a methyl-terminated silane, followed by adsorption of F108 to hydrophobic domains, and unmasking of coated glass (photoresist lift-off). Cell attachment on substrates resulted in non-adhesive donuts as seen in FIG. 8E (rather than continuous lanes seen in 6B, also visible in FIG. 4A).

### EXAMPLE 7

In order to confirm the role of PEO (rather than methylation of glass surface) in deterring cell adhesion on glass, methylated surfaces were compared to methylated surfaces following P108 treatment. Interestingly, methylation itself deterred cell adhesion initially; however, in the presence of media with 10% serum and cells that are known to secrete ECM in the local environment, methylated regions were rapidly invaded (FIG. 8C). In contrast, methylation followed by F108 exposure retained non-adhesive characteristics similar to those seen in FIG. 6C (FIG. 8D). FIG. 8E depicts a low-magnification bright field image of a F108 treated surface based on the pattern seen in FIG. 8A. Note the reproducible deterrence of cell adhesion from FI08-modified regions.

To further localize F108 deposition, we combined our findings with microfluidic techniques to deliver fluidic solutions (i.e., method seen in FIG. 5A). Therefore, F108 was localized through a fluidic channel and followed by seeding of fibroblast suspension. FIG. 8E depicts a horizontal lane of fibroblasts adhered to a glass surface but deterred from repeating F108 domains. Therefore, the ability to fabricate hierarchical tissue architectures has been demonstrated through patterning of non-adhesive domains as well adhesive domains seen in FIG. 5D.

The following specific references, also incorporated herein by reference, are indicated in the above discussion and examples by the corresponding number generally within parentheses or brackets.

1. Healy, K. E., C. H. Thomas, A. Rezania, J. E. Kim, P. J. McKeown, B. Lom, and P. E. Hockberger, Kinetics of bone cell organization and mineralization on materials with patterned surface chemistry. Biomaterials, 1996.17(2): p. 195-208.

2. Pins, G. D., M. Toner, and J. R. Morgan, Microfabrication of an analog of the basal lamina: biocompatible membranes with complex topographies. Faseb J, 2000.14(3); p. 593-602.

3. Chen, C. S., M. Mrksich, S. Huang, G. M. Whitesides, and D. E. Ingber, Geometric control of cell life and death. Science, 1997.276(5317): p. 1425-8.

4. Bhatia, S. N., U. J. Balis, M. L. Yarmush, and M. Toner, Effect of cell-cell interactions in preservation of cellular phenotype; cocultivation of hepatocytes and nonparenchymal cells. Faseb J, 1999. 13(14): p. 1883-900.

5. Hammarback, J. A., J. B. McCarthy, S. L. Palm, L. T. Furcht, and P. C. Letourneau, Growth cone guidance by substrate-bound laminin pathways is correlated with neuron-to-pathway adhesivity. Dev Biol, 1988.126(1): p. 29-39.

6. Lom, B., K. E. Healy, and P. E. Hockberger, A versatile technique for patterning biomolecules onto glass coverslips. Journal of Neuroscience Methods, 1993. 50(3): p. 385-397.

7. Singhvi, R., A. Kumar, G. P. Lopez, G. N. Stephanopoulos, D. I. Wang, G. M. Whitesides, and D. E. Ingber, Engineering cell shape and function. Science, 1994. 264(5159): p. 696-8.

8. Clemence, J. F., J. P. Ranieri, P. Aebischer, and H. Sigrist, Photoimmobilization of a bioactive laminin fragment and pattern-guided selective neuronal cell attachment. Bioconjug Chem, 1995. 6(4): p. 411-7.

9. Chen, G., Y. Ito, Y. Imanishi, A. Magnani, S. Lamponi, and R, Barbucci, Photoimmobilization of sulfated hyaluronic acid for antithrombogenicity. Bioconjug Chem, 1997. 8(5): p. 730-4.

10. Delamarche, E., A. Bernard, H. Schmid, B. Michel, and H. Biebuyck, Patterned delivery of immunoglobulins to surfaces using microfluidic networks. Science, 1997.276(5313); p. 779-81.

11. Folch, A. and M. Toner, Cellular micropatterns on biocompatible materials. Biotechnology Progress, 1998.14(3): p 388-392.

12. Chiu, D. T., N. L. Jeon, S. Huang, R. S. Kane, C. J. Wargo, I. S. Choi, D. E. Ingber, and G. M. Whitesides, Patterned deposition of cells and proteins onto surfaces by using three-dimensional microfluidic systems. Proc Natl Acad Sci USA, 2000. 97(6): p. 2408-13.

13. Odde, D. J. and M. J. Renn, Laser-guided direct writing of living cells. Biotechnology and Bioengineering, 2000. 67(3): p. 312-318.

14. Britland, S., E. Perez-Arnaud, P. Clark, B. McGinn, P. Connolly, and G. Moores, Micropatterning proteins and synthetic peptides on solid supports: a novel application for microelectronics fabrication technology. Biotechnol Prog, 1992. 8(2): p.155-60.

15. Bhatia, S. N., M. L. Yannush, and M. Toner, Micropatterning cells in tissue engineering, in Methods in Molecular Medicine; Tissue engineering methods and protocols, J. R. Morgan and M. Yarmush, Editors. 1998, Humana Press: New Jersey. p. 349-363.

16. Bhatia, S. N., M. L. Yarmush, and M. Toner, Controlling cell interactions by micropatterning in co-cultures: hepatocytes and 3T3 fibroblasts. J Biomed Mater Res, 1997.34(2): p. 189-99.

17. Folch, A., O. Hurtado, M. Schmidt, and M. Toner, Molding of deep polydimethylsiloxane microstructures for microfluidics and biologic applications. Journal of Biomechanical Engineering, 1999.121(1): p. 28-34.

18. Li, J. and K. Caldwell, Plasma protein interactions with Pluronic-treated colloids. Colloids and Surfaces B: Biointerfaces, 1996.7: p. 9-22.

19. Gombotz, W. R., G. H. Wang, T. A. Horbett, and A. S. Hoffiman, Protein adsorption to poly(ethylene oxide) surfaces. J Biomed Mater Res, 1991.25(12): p. 1547-62.

20. Desai, N. and J. Hubbell, Surface modifications of polymeric biomaterials for reduced thrombogenecity. Polym Mater Sci Eng, 1990.62: p. 731-735.

21. Lee, J., J. Kopecek, and J. Andrade, Surface properties of aqueous PEO-containing block copolymer surfactants: Protein resistant surfaces. Polym Mater Scie Eng, 1987, 57; p. 613-617.

22. Irvine, D. J., A. M. Mayes, S. K. Satija, J. G. Barker, S. J. Sofia-Allgor, and L. G. Griffith, Comparison of tethered star and linear poly(ethylene oxide) for control of biomaterials surface properties. J Biomed Mater Res, 1998. 40(3); p. 498-509.

23. Jo, S. and K. Park, Surface modification using silanated poly(ethylene glycol)s. Biomaterials, 2000.21(6): p. 605-16.

24. Klee, Surface Modification of Polyurethane by Plasma-Induced Graft Polymerization of Poly(ethylene glycol) Methacrylate*, Journal of Applied Polymer Science, 1996 61; p. 2373-2382.

25. Neff, J. A., K. D. Caldwell, and P. A. Tresco, A novel method for surface modification to promote cell attachment to hydrophobic substrates. J Biomed Mater Res, 1998, 40(4); p. 511-9.

26. Prime, K. and G. Whitesides, Adsorption of proteins onto surfaces containing end-attached oligo(ethylene oxide); A model system using self-assembled monolayers. J. Am. Chem. Soc, 1993.115: p. 10714-10721.

27. Harris, J. M., Poly(ethylene glycol) chemistry, biotechnical and biomedical applications. Topics in applied chemistry. 1992, New York: Plenum Press. xxi, 385.

28. Beebe, D. J., J. S. Moore, J. M. Bauer, Q. Yu, R. H. Liu, C. Devadoss, and B. H. Jo, Functional hydrogel structures for autonomous flow control inside microfluidic channels. Nature, 2000. 404(6778): p. 588,589-590.

29. Hubbell, Surface Physical Intepenetrating Networks of Poly(ehtyleneterephthalate) and Poly (ethylene oxide) with Biomedical Applications. Macromolecules, 1992, 25: p. 226-232.

30. Bearinger, J. P., D. G. Castner, S. L. Golledge, A. Rezania, S. Hubchak, and K. E. Healy, P(AAm-co-EG) interpenetrating polymer networks grafted to oxide surfaces: Surface characterization, protein adsorption, and cell detachment studies. Langmuir, 1997.13(19): p. 5175-5183.

31. Singhvi, R., G. Stephanopoulos, and D. I. C. Wang, Effects of Substratum Morphology On Cell Physiology—Review. Biotechnology and Bioengineering, 1994, 43(8): p. 764-771.

32. Saneinejad, S. and M. S. Shoichet, Patterned glass surfaces direct cell adhesion and process outgrowth of primary neurons of the central nervous system. J Biomed Mater Res, 1998. 42(1): p. 13-9.

33. Neff, J. A., P. A. Tresco, and K. D. Caldwell, Surface modification for controlled studies of cell-ligand interactions [In Process Citation]. Biomaterials, 1999. 20(23-24): p. 2377-93.

34. Bhatia, S. N., U. J. Balis, M, L. Yarmush, and M. Toner, Probing heterotypic cell interactions: hepatocytefunction in microfabricated co-cultures. J Biomater Sci Polym Ed, 1998. 9(11): p. 1137-60.

35. Bhatia, S. N., U. J. Balis, M. L. Yarmush, and M. Toner, Microfabrication of hepatocyte/fibroblast co-cultures: role of homotypic cell interactions. Biotechnol Prog, 1998. 14(3): p. 378-87.

36. Rotem, A., M. Toner, S. Bhatia, B. D. Foy, R. G. Tompkins, and M. L. Yarmush, Oxygen is a factor determining in vitro tissue assembly: Effects on attachment and spreading of hepatocytes. Biotechnology and Bioengineering, 1994. 43(7): p. 654-660.

37. Seglen, P. O., Preparation of isolated rat liver cells. Methods Cell Biol, 1976. 13; p. 29-83.

38. Dunn, J. C, M. L. Yarmush, H. G. Koebe, and R. G. Tompkins, Hepatocyte function and extracellular matrix geometry; long-term culture in a sandwich configuration [published erratum appears in FASEB J 1989 May; 3(7);1873]. Faseb J, 1989. 3(2): p.174-7.

39. Thomas, C. H., L. B. Lhoest, D. G. Castner, C. D. McFarland, and K. E. Healy, Surfaces designed to control the projected area and shape of individual cells. Transactions of the ASME, Journal of Biomechanical Engineering, 1999, 121(1): p. 40-8.

40. Spargo, B. J., M. A. Testoff, T. B. Nielsen, D. A. Stenger, J. J. Hickman, and A. S. Rudolph, Spatially controlled adhesion, spreading, and differentiation of endothelial cells on self-assembled molecular monolayers. Proc Natl Acad Sci USA, 1994. 91(23): p. 11070-4.

41. Bhatia, S. and C. Chen, Tissue Engineering at the Micro-Scale. Biomedical Microdevices, 1999, 2(2): p.131-44.

42. Britland, S., P. Clark, P. Connolly, and G. Moores, Micropatterned Substratum Adhesiveness—a Model For Morphogenetic Cues Controlling Cell Behavior, Experimental Cell Research, 1992. 198(1): p. 124-129.

43. Patel, N., et al., Spatially con trolled cell engineering on biodegradable polymer surfaces. Faseb J, 1998.12(14): p. 1447-54.

44. Balis, U., K. Behnia, B. Dwarkanath, S. Bhatia, S. Sullivan, M. Yarmush, and M. Toner, Oxygen Consumption Characteristics of Porcine Hepatocytes. Metabolic Engineering, 1999.1: p. 49-62.

45. Grinnell, F. and M. K. Feld, Fibronectin adsorption on hydrophilic and hydrophobic surfaces detected by antibody binding and analyzed during cell adhesion in serum-containing medium. Journal of Biological Chemistry, 1982. 257(9): p. 4888-93.

46. Grinnell, F., Fibronectin adsorption on material surfaces. Annals of the New York Academy of Sciences, 1987. 516(6): p. 280-90.

47. BASF, Pluronic and Tetronic Surfactants, 1990, BASF Co: NJ.

48. Thomas, C. H., C. D. McFarland, M. L. Jenkins, A. Rezania, J. G. Steele, and K. E. Healy, The role of vitronectin in the attachment and spatial distribution of bone-derived cells on materials with patterned surface chemistry. J Biomed Mater Res, 1997. 37(1): p. 81-93.

49. Steele, J. G., B. A. Dalton, G. Johnson, and P. A. Underwood, Polystyrene chemistry affects vitronectin activity; an explanation for cell attachment to tissue culture polystyrene but not to unmodified polystyrene. Journal of Biomedical Materials Research, 1993. 27(7): p. 927-40.

50. Amiji, M. and K. Park, Surface modification by radiation-induced grafting of peo/ppo/peo triblock copolymers. Journal of colloid and interface science, 1993. 155: p. 231-255.

51. Barnes, T. and C. Prestidge, PEO-PPO-PEO Block Copolymers at the Emulsion Droplet-Water Interface, Langmuir, 2000. Published on Web: p. 1-6.

52. McPherson, T., A. Kidane, I. Szleifer, and K. Park, Prevention of protein adsorption by tethered poly(ethylene oxide) layers: Experiments and single-chain mean-field analysis. Langmuir, 1998.14(1): p. 176-186.

53. Mrksich, M., C. S. Chen, Y. Xia, L. E. Dike, D. E. Ingber, and G. M. Whitesides, Controlling cell attachment on contoured surfaces with self-assembled monolayers of alkanethiolates on gold. Proc Natl Acad Sci USA, 1996. 93(20): p.10775-8.

54. Tien, J. and C. Chen, Personal Communication, Johns Hopkins University, 2000.

55. Klempner, D., L. Sperling, and L. Utracki, Interpenetrating polymer networks. Vol. IV.1994, NY: American Chemical Society.

56. Desai, N. P. and J. A. Hubbell, Surface Physical Interpenetrating Networks of Poly(Ethylene Terephthalate) and Poly(Ethylene Oxide) With Biomedical Applications, Macromolecules, 1992.25(1): p. 226-232.

57. Tseng, Y. C., T. McPherson, C. S. Yuan, and K. Park, Grafting of Ethylene Glycol-Butadiene Block Copolymers Onto Dimethyl-Dichlorosilane-Coated Glass By Gamma-Irradiation, Biomaterials, 1995.16(13): p. 963-972.

58. Jeong, B., Y. H. Bae, and S. W. Kim, In situ gelation of PEG-PLGA-PEG triblock copolymer aqueous solutions and degradation thereof J. Biomed Mater Res, 2000. 50(2): p.171-7.

59. Dori, Y., H. Bianco-Peled, S. K. Satija, G. B. Fields, J. B. McCarthy, and M. Tirrell, Ligand accessibility as means to control cell response to bioactive bilayer membranes. Journal of Biomedical Materials Research, 2000.50(1): p, 75-81.

60. James, C. D., R. C. Davis, L. Kam, H. G. Craighead, M. Isaacson, J. N. Turner, and W. Shain, Patterned protein layers on solid substrates by thin stamp microcontact printing. Langmuir, 1998.14(4): p. 741-744.

61. Bernard, A., E. Delamarche, H. Schmid, B. Michel, H. R. Bosshard, and H. Biebuyck, Printing patterns of proteins. Langmuir, 1998.14(9): p. 20225-2229.

62. Martin, B. D., B. P. Gaber, C. H. Patterson, and D. C. Turner, Direct protein microarray fabrication using a hydrogel “stamper”, Langmuir, 1998.14(15): p. 3971-3975.

63. Unger, M. A., H. P. Chou, T. Thorsen, A. Scherer, and S. R. Quake, Monolithic microfabricated valves and pumps by multilayer soft lithography. Science, 2000. 288(5463): p. 113-116.

64. Kapur, R., K. Giuliano, M. Campana, T. Adams, K. Olson, D. Jung, M. Mrksich, C. Vasudevan, and D. Taylor, Streamlining the Drug Discovery Process by Integrating Minitiaturization, High Throughput Screening, High Content Screening, and Automation on the CellChip System. Biomedical Microdevices, 1999.2(2); p. 99-110.

The invention has been described in detail with particular references to the preferred embodiments thereof. However, it will be appreciated that modifications and improvements within the spirit and scope of this invention may be made by those skilled in the art upon considering the present disclosure.

We claim:

1. A method of adhering a biomolecule to a substrate, comprising treating the substrate with 1) a surfactant compound and 2) a biomolecule.

2. The method of claim 1 wherein the surfactant compound is not covalently linked to the substrate.

3. The method of claim 1 or 2 wherein the surfactant compound comprises one or more hydrophobic regions and one or more hydrophilic regions.

4. The method of any one of claims 1 through 3 wherein the surfactant compound comprises one or more hetero atoms.

5. The method of any one of claims 1 through 4 wherein the surfactant compound comprises one or more alkoxy groups.

6. The method of any one of claims 1 through 5 wherein the surfactant is a polymeric material.

7. The method of any one of claims 1 through 6 wherein the surfactant has a molecular weight of at least about 1,000.

8. The method of any one of claims 1 through 7 wherein the surfactant is a polyalkyl oxide.

9. The method of any one of claims 1 through 8 wherein the surfactant comprises polyethylene oxide units.

10. The method of any one of claims 1 through 9 wherein the surfactant comprises polyC₃₋₂₀alkyl oxide units.

11. The method of any one of claims 1 through 9 wherein the surfactant comprises polyC₃₋₁₂alkyl oxide units.

12. The method of any one of claims 1 through 9 wherein the surfactant comprises polypropylene oxide units.

13. The method of any one of claims 1 through 12 wherein the surfactant comprises thiol groups.

14. The method of any one of claims 1 through 12 wherein the surfactant comprises alkylthio groups.

15. The method of any one of claims 1 through 14 wherein the surfactant comprises charged or chargeable groups.

16. The method of any one of claims 1 through 12 wherein the surfactant is a Pluronic or Tween material.

17. The method of any one of claims 1 through 17 wherein the surfactant is in a defined patterned on the substrate, with selective substrate areas bared of the surfactant.

18. The method of any one of claims 1 through 17 wherein the biomolecule resides on substrate areas bared of the surfactant.

19. The method of any one of claims 1 through 18 wherein an applied layer of the surfactant is exposed to patterned radiation to define a desired pattern of the surfactant layer.

20. The method of claim 19 wherein the biomolecule selectively adheres to substrate regions bared of the surfactant through the exposure.

21. The method of any one of claims 1 through 20 wherein a network of microchannels positioned on the substrate define selected areas of deposition of the biomolecule.

22. The method of any one of claims 1 through 16 wherein a selected pattern of the surfactant or biomolecule is defined by physical treatment of the substrate.

23. The method of claim 22 wherein the physical treatment comprises a stamping process, microfluidics, photolithography, microcontact printing, nanopen lithography, subtraction active devices or eletrophoresis.

24. The method of any one of claims 1 through 23 wherein the substrate is treated with a binding agent prior to treating with the biomolecule.

25. The method of claim 24 wherein the binding agent comprises a protein.

26. The method of any one of claims 1 through 25 wherein the biomolecule is selected from peptides, polypeptides, nucleic acids, nucleic acid binding partners, proteins, receptors, antibodies, enzymes, carbohydrates, oligo saccharides, polysaccharides, cells, cell aggregagates, cell components, lipids, arrays of ligands (e.g. non-protein ligands), liposomes, or microorganisms, e.g., bacteria, viruses.

27. The method of claim 24 or 25 wherein cells bind to the binding agent.

28. The method of claim 27 wherein the cells comprise bacterial cells, mammalian cells such as chinese hamster ovary (CHO), baby hamster kidney (BHK), COS, human fibroblast, hematopoietic stem cells, hepatocytes, and hybridoma cell lines; yeast; fungi; and cell lines useful for expression systems such as yeast or Xenopus laevis oocytes.

29. The method of claims 1-28, further comprising providing at least one additional and different biomolecule.

30. The method of any one of claims 1 through 29 wherein the substrate surface comprises a polymer.

31. The method of any one of claims 1 through 30 wherein the substrate surface comprise glass.

32. A method of adhering a biomolecule to a substrate, comprising:
a) providing a binding agent onto a template having a desired pattern;
b) contacting the template with the substrate so that the binding agent is transferred to the substrate in a pattern corresponding to the template;
c) providing a non-adhesive agent to the substrate having the bindging agent pattern thereon, wherein the non-adhesive agent adheres to the substrate area not comprising the binding agent;
d) providing biomolecules to the substrate, wherein the biomolecules adhere to the binding agent but not the non-adhesive agent.

33. The method of claim 32 wherein the binding agent comprises a protein capable of adhering to the biomolecule.

34. The method of claim 32 or 33 wherein the non-adhesive agent comprises a surfactant compound.

35. The method of any one of claims 32 through 34 wherein the biomolecule are cells.

36. The method of claims 32 through 35, further comprising providing at least one additional and different biomolecule.

37. A method of adhering a biomolecule to a substrate comprising:
a) providing a surfactant onto template;
b) contacting the template with the substrate so that the surfactant is transferred to the substrate in a pattern corresponding to the template;
c) providing a binding agent to the substrate having the surfactant pattern thereon, wherein the binding agent adheres to the substrate area not comprising the surfactant;
d) providing a non-adhesive agent to the surface having the pattern of hydrophilic and hydrophobic agents thereon;
e) providing a binding agent, wherein the binding agent binds to the hydrophilic agent;
f) providing biomolecules to the surface, wherein the biomolecules adhere to binding agent but not the non-adhesive agent.

38. The method of claim 37 wherein the binding agent comprises a protein capable of adhering to the biomolecule.

39. The method of any one of claims 36 and 37 wherein the biomolecule is a cell.

40. The method of any one of claims 36 through 39, further comprising providing at least one additional and different biomolecule.

41. A method of patterning a surface with biomolecules comprising:
a) providing a mask to the surface, wherein the mask has a desired pattern of open areas and closed areas;
b) providing a non-adhesive agent to the surface;
c) providing a binding agent;
d) providing biomolecules to the surface;
wherein the biomolecules adhere to binding agent but not the non-adhesive agent.

42. The method of claim 41 wherein the binding agent comprises a protein capable of adhering to the biomolecule.

43. The method of claim 41 or 42 wherein the non-adhesive agent comprises a surfactant compound.

44. The method of any one of claims 41 through 43 wherein the biomolecule is a cell.

45. The method of claims 41 through 44, further comprising providing at least one additional and different biomolecule.

46. A device for adhering a biomolecule in a predetermined position comprising:
a substrate having thereon a plurality of cytophilic regions that can adhere a biomolecule on the substrate by cytophobic regions to which the biomolecules do not adhere contiguous with the cytophilic regions, wherein the cytophobic regions comprise one or more surfactant compounds.

47. The device of claim 46 wherein the surfactant compound is not covalently linked to the substrate.

48. The device of claim 46 or 47 wherein the surfactant compound comprises one or more hydrophobic regions and one or more hydrophilic regions.

49. The device of any one of claims 46 through 48 wherein the surfactant compound comprises one or more hetero atoms.

50. The device of any one of claims 46 through 48 wherein the surfactant compound comprises one or more alkoxy groups.

51. The device of any one of claims 46 through 50 wherein the surfactant is a polymeric material.

52. The device of any one of claims 46 through 51 wherein the surfactant has a molecular weight of at least about 1,000.

53. The device of any one of claims 46 through 51 wherein the surfactant has a molecular weight of at least about 10,000.

54. The device of any one of claims 46 through 53 wherein the surfactant comprises polyethylene oxide units.

55. The device of any one of claims 46 through 54 wherein the surfactant comprises polyC₃₋₂₀alkyl oxide units.

56. The device of any one of claims 46 through 54 wherein the surfactant comprises polypropylene oxide units.

57. The device of any one of claims 46 through 56 wherein the surfactant comprises thiol groups.

58. The device of any one of claims 46 through 56 wherein the surfactant comprises alkylthio groups.

59. The device of any one of claims 46 through 58 wherein the surfactant comprises charged or chargeable groups.

60. The device of any one of claims 46 through 56 wherein the surfactant is a Pluronic or Tween material.

61. The device of any one of claims 46 through 60 wherein the substrate is a microarray substrate.

62. The device of any one of claims 46 through 61 wherein the substrate comprises at least 1 million biomolecules per cm².

63. The device of any one of claims 46 through 61 wherein the substrate comprises at least 2 million biomolecules per cm².
